SEQUENCE LISTING (197 sequences)

SEQ ID NO: 1 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
  REPEAT 1..3
GPPGPPGPPGPPGPPGPPGPPGPPGPP

SEQ ID NO: 2 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 3 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 4 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 5 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 6 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 7 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 8 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 9 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 10 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 11 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 12 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 13 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 14 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 15 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 16 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 17 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 18 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 19 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 20 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 21 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 22 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 23 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 24 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 25 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 26 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 27 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 28 - protein, 27 aa, synthetic construct
  MOD_RES 3: note = trans-hydroxyproline (2S, 4R-hydroxyproline)
  MOD_RES 6: note = trans-hydroxyproline (2S, 4R-hydroxyproline)
  MOD_RES 9: note = trans-hydroxyproline (2S, 4R-hydroxyproline)
  MOD_RES 12: note = trans-hydroxyproline (2S, 4R-hydroxyproline)
  MOD_RES 15: note = trans-hydroxyproline (2S, 4R-hydroxyproline)
  MOD_RES 18: note = trans-hydroxyproline (2S, 4R-hydroxyproline)
  MOD_RES 21: note = trans-hydroxyproline (2S, 4R-hydroxyproline)
  MOD_RES 24: note = trans-hydroxyproline (2S, 4R-hydroxyproline)
  MOD_RES 27: note = trans-hydroxyproline (2S, 4R-hydroxyproline)
GPPGPPGPPGPPGPPGPPGPPGPPGPP

SEQ ID NO: 29 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 30 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 31 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 32 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 33 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 34 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPOGPOGPOGPOGPOGPOGPOGPOGPO

SEQ ID NO: 35 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 36 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 37 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 38 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 39 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 40 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 41 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 42 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 43 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 44 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 45 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 46 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 47 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 48 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 49 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 50 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 51 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 52 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 53 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 54 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 55 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 56 - protein, 27 aa, synthetic construct
  MOD_RES 2: note = cis-fluoroproline (2S,4S-4-fluoroproline)
  MOD_RES 5: note = cis-fluoroproline (2S,4S-4-fluoroproline)
  MOD_RES 8: note = cis-fluoroproline (2S,4S-4-fluoroproline)
  MOD_RES 11: note = cis-fluoroproline (2S,4S-4-fluoroproline)
  MOD_RES 14: note = cis-fluoroproline (2S,4S-4-fluoroproline)
  MOD_RES 17: note = cis-fluoroproline (2S,4S-4-fluoroproline)
  MOD_RES 20: note = cis-fluoroproline (2S,4S-4-fluoroproline)
  MOD_RES 23: note = cis-fluoroproline (2S,4S-4-fluoroproline)
  MOD_RES 26: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 57 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 58 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 59 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 60 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 61 - protein, 27 aa, synthetic construct
  MOD_RES 3: note = trans-hydroxyproline (2S, 4R-hydroxyproline)
GPPGPPGPPGPPGPPGPPGPPGPPGPP

SEQ ID NO: 62 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 63 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 64 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 65 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 66 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 67 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 68 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 69 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 70 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 71 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 72 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 73 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 74 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 75 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 76 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 77 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 78 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 79 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 80 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 81 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 82 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 83 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 84 - protein, 27 aa, synthetic construct
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 85 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 86 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..2: note = Repeat 9 times
GPPGPPGPPGPPGPPGPPGPPGPPGPP

SEQ ID NO: 87 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 88 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = trans-hydroxyproline (2S, 4R-hydroxyproline)
GPPGPPGPPGPPGPPGPPGPPGPPGPP

SEQ ID NO: 89 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 90 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 91 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 92 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 93 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 94 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 95 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 96 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 97 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 98 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 99 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 100 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 101 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 102 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 103 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 104 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 105 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 106 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 107 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 108 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 109 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 110 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 111 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 112 - protein, 27 aa, synthetic construct
  MOD_RES 2: note = cis-fluoroproline (2S,4S-4-fluoroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 113 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 114 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 115 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 116 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 117 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 118 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 119 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 120 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 121 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 122 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REGION 1..3: note = Repeat 9 times
GPPGPPGPPGPPGPPGPPGPPGPPGPP

SEQ ID NO: 123 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 124 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 125 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 126 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 127 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 128 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 129 - protein, 27 aa, synthetic construct
  MOD_RES 3: note = trans-hydroxyproline (2S, 4R-hydroxyproline)
  REPEAT 1..3: note = Repeat 9 times
  MOD_RES 2: note = cis-chloroproline (2S,4S-4-chloroproline)
GPPGPPGPPGPPGPPGPPGPPGPPGPP

SEQ ID NO: 130 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 131 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 132 - protein, 27 aa, synthetic construct
  MOD_RES 2: note = cis-chloroproline (2S,4S-4-chloroproline)
  MOD_RES 5: note = cis-chloroproline (2S,4S-4-chloroproline)
  MOD_RES 8: note = cis-chloroproline (2S,4S-4-chloroproline)
  MOD_RES 11: note = cis-chloroproline (2S,4S-4-chloroproline)
  MOD_RES 14: note = cis-chloroproline (2S,4S-4-chloroproline)
  MOD_RES 17: note = cis-chloroproline (2S,4S-4-chloroproline)
  MOD_RES 20: note = cis-chloroproline (2S,4S-4-chloroproline)
  MOD_RES 23: note = cis-chloroproline (2S,4S-4-chloroproline)
  MOD_RES 26: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 133 - protein, 27 aa, synthetic construct
  REPEAT 1..3: note = Repeat 9 times
  MOD_RES 2: note = cis-chloroproline (2S,4S-4-chloroproline)
  MOD_RES 3: note = trans-hydroxyproline (2S, 4R-hydroxyproline)
GPPGPPGPPGPPGPPGPPGPPGPPGPP

SEQ ID NO: 134 - protein, 27 aa, synthetic construct
  METAL 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 135 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 136 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 137 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REGION 1..3: note = Repeat 9 times
GPPGPPGPPGPPGPPGPPGPPGPPGPP

SEQ ID NO: 138 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 139 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 140 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 141 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 142 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 143 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 144 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 145 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 146 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 147 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 148 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 149 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 150 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 151 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 152 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 153 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 154 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 155 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 156 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 157 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 158 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 159 - protein, 27 aa, synthetic construct
  MOD_RES 2: note = cis-chloroproline (2S,4S-4-chloroproline)
  MOD_RES 3: note = cis-chloroproline (2S,4S-4-chloroproline)
  REPEAT 1..3: note = Repeat 9 times
GPPGPPGPPGPPGPPGPPGPPGPPGPP

SEQ ID NO: 160 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 161 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 162 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 163 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 164 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 165 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 166 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 167 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 168 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3: note = Repeat 9 times
GPPGPPGPPGPPGPPGPPGPPGPPGPP

SEQ ID NO: 169 - protein, 27 aa, synthetic construct
  MOD_RES 2: note = cis-chloroproline (2S,4S-4-chloroproline)
  MOD_RES 3: note = trans-hydroxyproline (2S, 4R-hydroxyproline)
  REPEAT 1..3
GPPGPPGPPGPPGPPGPPGPPGPPGPP

SEQ ID NO: 170 - protein, 36 aa, synthetic construct
GPPGPPGPPGPPGPPGFPGERGPPGPPGPPGPPGPP

SEQ ID NO: 171 - protein, 36 aa, synthetic construct
GPPGPPGPPGPPGPPGLOGERGPPGPPGPPGPPGPP

SEQ ID NO: 172 - protein, 36 aa, synthetic construct
GPPGPPGPPGPPGPPGROGERGPPGPPGPPGPPGPP

SEQ ID NO: 173 - protein, 36 aa, synthetic construct
GPPGPPGPPGPPGPPGAOGERGPPGPPGPPGPPGPP

SEQ ID NO: 174 - protein, 36 aa, synthetic construct
GPPGPPGPPGPPGPPGFOGERGPPGPPGPPGPPGPP

SEQ ID NO: 175 - protein, 36 aa, synthetic construct
GPPGPPGPPGPPGPPGLOGENGPPGPPGPPGPPGPP

SEQ ID NO: 176 - protein, 36 aa, synthetic construct
GPPGPPGPPGPPGPPGVOGEAGPPGPPGPPGPPGPP

SEQ ID NO: 177 - protein, 36 aa, synthetic construct
GPPGPPGPPGPPGPPGFPGENGPPGPPGPPGPPGPP

SEQ ID NO: 178 - protein, 36 aa, synthetic construct; identical to SEQ ID NO: 170

SEQ ID NO: 179 - protein, 39 aa, synthetic construct
GPPGPPGPPGPPGPPGLOGERGROGPPGPPGPPGPPGPP

SEQ ID NO: 180 - protein, 36 aa, synthetic construct; identical to SEQ ID NO: 174

SEQ ID NO: 181 - protein, 36 aa, synthetic construct; identical to SEQ ID NO: 172

SEQ ID NO: 182 - protein, 36 aa, synthetic construct; identical to SEQ ID NO: 173

SEQ ID NO: 183 - protein, 39 aa, synthetic construct; identical to SEQ ID NO: 179

SEQ ID NO: 184 - protein, 36 aa, synthetic construct; identical to SEQ ID NO: 171

SEQ ID NO: 185 - protein, 36 aa, synthetic construct; identical to SEQ ID NO: 174

SEQ ID NO: 186 - protein, 36 aa, synthetic construct; identical to SEQ ID NO: 177

SEQ ID NO: 187 - protein, 36 aa, synthetic construct; identical to SEQ ID NO: 170

SEQ ID NO: 188 - protein, 36 aa, synthetic construct; identical to SEQ ID NO: 174

SEQ ID NO: 189 - protein, 36 aa, synthetic construct; identical to SEQ ID NO: 177

SEQ ID NO: 190 - protein, 36 aa, synthetic construct; identical to SEQ ID NO: 170

SEQ ID NO: 191 - protein, 36 aa, synthetic construct
GPPGPPGPPGPPGPPGXKGHRGPPGPPGPPGPPGPP

SEQ ID NO: 192 - protein, 42 aa, synthetic construct
GPPGPPGPPGPPGPPGXRHQOGVMGFOGPPGPPGPPGPPGPP

SEQ ID NO: 193 - protein, 39 aa, synthetic construct
GPPGPPGPPGPPGPPGLOGQRGERGPPGPPGPPGPPGPP

SEQ ID NO: 194 - protein, 39 aa, synthetic construct
GPPGPPGPPGPPGPPGLOGMKGHRGPPGPPGPPGPPGPP

SEQ ID NO: 195 - protein, 38 aa, synthetic construct
GPPGPPGPPGPPGPPGPQGIAGQGPPGPPGPPGPPGPP

SEQ ID NO: 196 - protein, 38 aa, synthetic construct
GPPGPPGPPGPPGPPGPQGIWGQGPPGPPGPPGPPGPP

SEQ ID NO: 197 - protein, 45 aa, synthetic construct
GPPGPPGPPGPPGPPGPLGIAGITGARGLAGPPGPPGPPGPPGPP

(19) United States
(12) Patent Application Publication
(10) Pub. No.: US 2024/0400647 A1
(43) Pub. Date: Dec. 5, 2024

(54) Biologically Active Collagen Hybridizing Peptides

(71) Applicant: 3HELIX, INC., Salt Lake City, UT (US)

(72) Inventors: Lucas BENNINK, Salt Lake City, UT (US); Michael KIRKNESS, Salt Lake City, UT (US); Julian KESSLER, Salt Lake City, UT (US); Michael YU, Salt Lake City, UT (US)

(73) Assignee: 3HELIX, INC., Salt Lake City, UT (US)

(21) Appl. No.: 18/698,649

(22) PCT Filed: Oct. 7, 2022
(86) PCT No.: PCT/US2022/077730

§ 371 Date: Apr. 4, 2024

Related U.S. Application Data
(60) Provisional application No. 63/253,473, filed on Oct. 7, 2021.

(51) Int. Cl. C07K 14/78; A61K 8/64; A61Q 19/00; C07K 14/705

(52) U.S. Cl. CPC C07K 14/78; A61K 8/64; A61Q 19/00; C07K 14/705

(57) ABSTRACT

A modified collagen hybridizing peptide (CHP) may include one or more binding partners crosslinked to a CHIP. The CHP has a sequence represented by Formula (I): (Gly-X-Y)_{a-b}, wherein Gly is glycine, at least one of X and Y is proline, modified proline and/or hydroxy proline, and a is 3 and b is 20. The one or more binding partners is selected from the group consisting of integrin sites, integrin binding sites, crosslinking sites, von Willebrand Factor (VWF) discoidin domain receptor (DDR) 1, DDR 2, SPARC binding peptides, fibronectin binding peptides, engineered integrin binding peptides, matrix metalloproteinase (MMP) cleavage sites. Cathepsin K (CATK) sites, and osteoclast-associated receptors (OSCAR).

### BACKGROUND

Collagen homeostasis is a complex process which involves both the synthesis and degradation of collagen. Collagen homeostasis is tightly controlled mainly via a specialized group of cells called fibroblasts. Fibroblasts produce both collagen and matrix metalloproteinases (MMPs) in response to chemical signals, cell to extracellular matrix (ECM) interactions, environmental conditionals like UV light and force. The most abundant ECM protein is collagen. Collagen has binding sequences present that are required to be in triple helical form to be recognized by fibroblasts, MMPs, Cathepsin K or other cell types including integrins, discoidin domain receptors, osteoclast-associated receptors (OSCAR) and many others. Importantly fibroblasts respond to mechanical signals through their interactions with collagen mediated by integrins.

Many events can cause fibroblasts to decrease their production of collagen such as diseases like osteoarthritis; natural events like aging (senescent fibroblast aging); decreased mechanical stimulation; environmental factors like UV damage, or chemical signals like reactive oxygen species. Decreased collagen synthesis often causes a reduction in mechanical stimulation as there is less collagen around for the fibroblasts to interact with. This decreased mechanical stimulation can cause an increase in the matrix metalloproteinase (MMP) production and, in essence, create a negative feedback loop where the reduced mechanical stimulation causes increased MMP production and a decrease of collagen production which again decreases the mechanical stimulation. This can occur with dermal fibroblasts in aged skin, whether it is from increased UV exposure or natural aging. A similar process can occur to mechanically damaged collagen containing tissues like tendons or cartilage.

One can push the fibroblasts out of this negative feedback loop by providing additional binding sites for fibroblasts to bind to. Offering new mechanical cues to the microenvironment will increase collagen production thereby increasing mechanical stimulation for the cells in order to create a positive feedback loop, encouraging tissue regeneration and repair. Therefore, methods and compositions that can stimulate fibroblasts to increase collagen production are desired to restore structural integrity of the ECM and repair damage to collagen containing tissues.

### SUMMARY OF THE DISCLOSURE

In one aspect, to solve the problem of stimulating fibroblasts to modulate the production of ECM components such as collagen and MMPs, the present disclosure provides a modified collagen hybridizing peptide (CHP) that includes one or more binding partners crosslinked to a CHP. The CHP has a sequence represented by Formula I: (Gly-X-Y)_{a-b}, wherein Gly is glycine, at least one of X and Y is proline, modified proline and/or hydroxy proline, and a is 3 and b is 20. The one or more binding partners is selected from the group consisting of integrin sites, integrin binding sites, crosslinking sites, von Willebrand Factor (VWF), discoidin domain receptor (DDR) 1, DDR2, SPARC binding peptides, fibronectin binding peptides, engineered integrin binding peptides, matrix metalloproteinase (MMP) cleavage sites, Cathepsin K (CATK) sites, and osteoclast-associated receptors (OSCAR).

In one aspect, to solve the problem of increasing collagen production in a subject, the present disclosure provides a composition including a modified collagen hybridizing peptide (CHP) disclosed herein.

In one aspect, to solve the problem of increasing collagen degradation in a subject, the present disclosure provides a composition including a modified collagen hybridizing peptide (CHP) disclosed herein.

In one aspect, to solve the problem of increasing MMP production in a subject, the present disclosure provides a composition including a modified collagen hybridizing peptide (CHP) disclosed herein.

In one aspect, to solve the problem of decreasing collagen production in a subject, the present disclosure provides a composition including a modified collagen hybridizing peptide (CHP) disclosed herein.

In one aspect, to solve the problem of decreasing MPP production in a subject, the present disclosure provides a composition including a modified collagen hybridizing peptide (CHP) disclosed herein.

In another aspect, the present disclosure further provides a method of increasing collagen production in a subject including administering a cosmetic composition including a modified collagen hybridizing peptide (CHP) disclosed herein to the subject.

In one aspect, to solve the problem of inducing dermal repair in a subject, the present disclosure provides a composition including a modified collagen hybridizing peptide (CHP) disclosed herein.

In another aspect, the present disclosure further provides a method of inducing dermal repair in a subject including administering a cosmetic composition including a modified collagen hybridizing peptide (CHP) disclosed herein to the subject.

In one aspect, to solve the problem of inducing tissue repair in a subject, the present disclosure provides a composition including a modified collagen hybridizing peptide (CHP) disclosed herein.

In another aspect, the present disclosure further provides a method of inducing tissue repair in a subject including administering a medical device composition including a modified collagen hybridizing peptide (CHP) disclosed herein to the subject.

### BRIEF DESCRIPTION OF THE DRAWINGS

FIG. 1A. Illustrates the bioACTIVE CHP, comprising of triple helical forming sections covalently linked to the bioACTIVE sequences.

FIG. 1B represents the unique binding mechanism of the bioACTIVE CHP. This allows the CHP to be inactive in its single chain conformation but becomes activated once it forms a triple helix with the damaged collagen, once in the triple helical form the collagen producing cells can bind and mechanically sense the newly formed triple helix.

FIG. 2A shows CHPs targeting and binding to damaged collagen induced by collagenase activity in an in vivo mouse model of tendonitis;

FIG. 2B shows a stark difference in CHP binding between a normal knee joint and an osteoarthritic (OA) knee joint in stained human tissue sections.

FIG. 2C shows increased CHP binding in an aged human tissue section of skin compared to that of the young tissue.

FIG. 3A shows a first derivative from the thermal melting curve (Tm) of the bioACTIVE CHPs disclosed herein. The Tm was taken from 4° C. to 80° C. at a rate of 0.5° C. per minute and gives the thermal transition temperature when the CHPs go from an ordered triple-helical structure to a more disorganized monomeric structure. The first derivative shows the inflection point of the Tm curve as the minimum of the graph. This point corresponds to the melting temperature of the CHP, i.e., they are the temperatures that are required to break up any homotrimeric triple-helix formed. This shows that the bioACTIVE CHPs have a Tm near body temperature (37° C.) making them ideal candidates for binding damaged collagen in the body. Each CHP was dissolved at 150 μM concentration in DI water. Prior to measuring the Tm, each CHP was allowed to fully fold by being placed in a refrigerator (4° C.) for 48 hrs.

FIG. 3B shows the average amount of the CHP binding in each bioactive Binding Site in B-G₃-(GfO) 5-Binding Site-(GfO) 5. A 10% gelatin solution (w/v) from porcine skin (Sigma-Aldrich 48724-100G-F) in 1×PBS was made. To keep this from gelating, the 10% gelatin solution was kept under constant heating (65° C.). Next, 25 μL of heated 10% gelatin solution was added to coat the bottom of a 96-well plate, then excess was immediately taken off leaving a thin layer of gelatin. The plate was allowed to cool at 4° C. for 15 minutes. Then, 100 μL of an EDC crosslinking solution consisting of 1×MES Buffer, EDC (1-ethyl-3-(3-dimethylaminopropyl) carbodiimide hydrochloride), NHS (N-hydroxysuccinimide) was added to the surface of each well and allowed to crosslinked overnight at 4° C. Crosslinking solution was removed, then each well was washed three times with 200 μL of 1×PBS. Next, 50 μL of CHPs were added at a concentration of 100 μM and allowed to bind overnight at 37° C. After binding, the wells were washed three times with 200 μL of 1×PBS and 20 μL of FITC-streptavidin fluorophore (0.01 mg/ml) was added to each well and allowed to bind for one hour at room temperature to visualize the amount of CHP binding.

FIG. 4A to 4C show images of the human dermal fibroblast (HDF) cells of healthy model (FIG. 4A), damaged model (FIG. 4B) and damaged model treated with bioactive CHPs (FIG. 4C) in 2.5D Gelatin Experiments. In the 2.5D gelatin experiment, a 10% gelatin solution was made from porcine skin (Sigma-Aldrich 48724-100G-F) in 1×PBS and kept at 65° C. to prevent gelation before application. Next, 250 μL of 10% gelatin solution was added to each corresponding well of a 24-well plate. The plate was allowed to cool at 4° C. for 15 minutes. Next, 400 μL of the EDC-crosslinking solution. Crosslinking solution was removed and wells were washed 3× using 400 μL of 1×PBS. Next, 70% EtOH was added to each well and the plates were placed under a UV light for 1 hour for sterilization. The wells were washed 5× with 1 mL of 1×PBS. CHPs were preheated at 80° C. for 15 minutes. A master mix of 15,000 Human Dermal Fibroblast (ATCC), 50 L at 20 μM CHP and 950 μL of Fibroblast Growth Media with 10% FBS was made for each well in each treatment. 1000 μL of this mix was added to each corresponding well. Wells were imaged at 24 hours on EVOS Brightfield 4× magnification. Images were analyzed in ImageJ software.

FIG. 4D shows results of HDF cell length in the 2.5D gelatin experiment by creating a 12-unit grid and collecting images after 24 hours incubation at 37° C. To reduce subjectivity in analyzing the cell length, grids were randomly selected and counted until 100 cells were analyzed per image. The individual cells were measured via the internal ImageJ measuring tool to determine cell length.

FIG. 4E shows the average cell count numbers after 24 hours incubation on 250 μL of 10% gelatin. The images were analyzed using ImageJ maxima finder to count the number of cells.

FIGS. 5A and 5B show values of delta CT fold change from PBS with ex vivo native models purchased from GenoSkin. Models passed all OC tests by GenoSkin. Samples were surgically removed from the abdomen of a female, 69 year-old, Caucasian with type 1 phototype skin (light skinned). Once ex vivo models were received, 1 mL of supplied GenoSkin media was added to each model and were allowed to calibrate at 37° C. for 1 hour. CHPs were heated to 80° C. for 15 min and then quickly quenched on ice (˜30-90 sec). Next, 100 μL of 100 μM CHP was added to 900 μL of media. Media was removed from each model and 1000 μL of fresh media+CHP was added to each well per treatment. CHPs were absorbed through the bottom of the models, simulating an in situ injection. The media+CHP mixture was removed and replenished daily. Seven days post-surgery (6 days of treatment), models were homogenized and lysed for RNA extraction following the Qiagen RNeasy Micro Kit protocol. RNA was quantified on nanodrop. Reverse transcription was performed following Qiagen QuantiTect Reverse Transcription Kit. RT-PCR was performed using Taqman reagents included Col1A1 as the gene of interest and 18S which was the housekeeping gene. Reported values are delta CT fold change from PBS wells.

FIG. 5C shows ELISA results of Pro-Collagen I in ex vivo hole punch wound healing models. The ex vivo hole punch models were purchased from GenoSkin. Samples were surgically removed from the abdomen of a female, 63 year-old, African American with type 5 phototype skin (dark skin). Once ex vivo models were received, 1 mL of supplied GenoSkin media was added to each model and were allowed to calibrate at 37° C. for 1 hour. GenoSkin Media was removed from each model and replaced with 1000 μL of fresh media each day. CHPs were heated at 80° C. for 15 min and then quickly quenched on ice (˜30-90 sec). Next, 100 μL of 100 μM CHP solution was applied to the top of the models and replaced every 24 hours. Media was collected each day then centrifuged, and aliquoted into 100 μl samples. These samples were stored at −80° C. Samples were then processed using the manufacturer's protocol for Pro-Collagen I ELISA Kits from Abcam (AB120966).

FIG. 6. shows changes in gene expression caused by bioactive CHP disclosed herein.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide conjugate is disclosed and discussed and a number of modifications that can be made to a number of molecules including the peptide conjugate are discussed, each and every combination and permutation of the peptide conjugate and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary unless a particular order is clearly indicated by the context.

As used herein, the term “about” refers to a numeric value, including, for example, whole numbers, fractions, and percentages, whether or not explicitly indicated. The term “about” generally refers to a range of numerical values (e.g., +/−5, 6, 7, 8, 9 or 10% of the recited value) that one of ordinary skill in the art would consider equivalent to the recited value (e.g., having the same function or result). In some instances, the term “about” may include numerical values that are rounded to the nearest significant figure. In general, the term “about” includes at least the values for a given quantity that fall within the corresponding tolerances for manufacture, formulation and/or measurement.

As used herein “collagen” can be from any tissue type (e.g., bone, dermis, tendon, ligaments, etc.). Collagen can refer to a molecule in which three alpha chains of polyproline II-like structure fold together into a triple helix. Additionally, this can apply to any protein that contains a triple-helical region including collagen types I-XXVIII and bacterial collagen. The term “collagen” as used herein can refer to all forms of collagen, including artificial collagen and collagen which has been processed or otherwise modified. In some embodiments, the collagen is selected from type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, and a combination thereof.

As used herein, the term “proline or modified proline” means the amino acid proline and various isomers, analogs and variants thereof, including both natural and non-natural isomers. In one example, the modified proline includes an electron withdrawing group. Examples of modified proline include, without limitation, hydroxyproline, methylated proline, 4-fluoro proline, and 4-chloroproline.

In some embodiments, the method excludes collecting a sample from the subject. In some embodiments, “subject” herein can refer to a human or an animal or bacteria or cell cultures from any of the aforementioned groups. Non-limiting examples of animals include vertebrates such as a primate, a rodent, a domestic animal, or a game animal. Primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, moose, feline species (e.g., domestic cat), and canine species (e.g., dog, fox, wolf). Fish including Chondrichthyes (cartilaginous fishes) and Osteichthyes (bony fishes). The subject may be mammal. The mammal can be a human, non-human primate, mouse, rat, dog, cat, horse, or cow, but are not limited to these examples. In addition, the methods described herein can be used to diagnose and/or treat domesticated animals or pets. The term does not denote a particular age or sex. Thus, adult and newborn subjects, as well as fetuses, whether male or female, are intended to be included within the scope of this term.

The term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a disease or injury involving collagen damage can refer to reducing or eliminating the amount of damaged/denatured collagen. Treatment can also be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### I. Introduction

With reference to the appended drawings, exemplary embodiments of the present disclosure will be described in detail below. To aid in understanding the present disclosure, like numbers refer to like elements throughout the description of the figures, and the description of the same elements will be not reiterated.

### II. Skin Aging

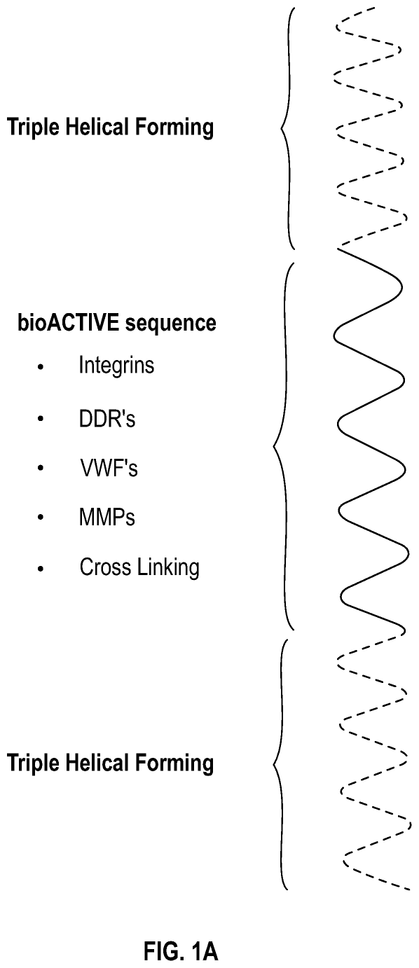

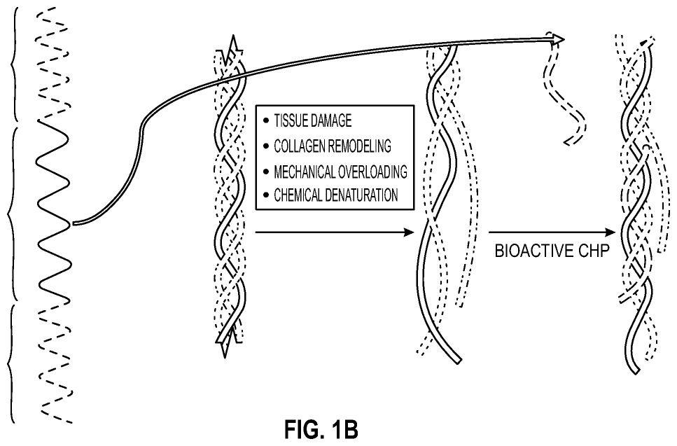

As we age, we have a decreased collagen production and increased MMP production in the skin causing wrinkled (and thin) skin. FIG. 1 illustrates the pathway for aging-induced production of reactive oxygen species (ROS) and MMPs in fibroblasts. ROS are produced initially because of exposure to external factors such as UV and pollution. Excess ROS further exacerbate the damage to the tissue by pushing young fibroblasts along a pathway of increased ROS and MMP production (negative feedback loop).

A loss of collagen and increased ROS levels in the ECM causes the dermal fibroblasts to become rounded and aged. This causes additional ROS production and MMP production and further decreases collagen production, thereby disrupting the collagen in the ECM that supports the fibroblasts in an extended “young” configuration. The loss of collagen further pushes the fibroblasts into the rounded “aged” configuration creating an accelerating negative feedback loop. A similar phenomenon occurs when a collagenous tissue such as tendons, for example, is damaged because of trauma (i.e., a tear). The high ROS levels and the disruption of collagen matrix resulting from the trauma accelerate the rounding of fibroblasts.

In order to reverse the effects of aging on the ECM or to repair a damaged collagenous tissue, the problem of providing mechanical stimulation to rounded fibroblasts needs to be solved.

### III. Stimulating Fibroblasts to Increase Collagen Production

In one aspect, to solve the problem of stimulating fibroblasts to increase collagen production, the present disclosure provides a modified collagen hybridizing peptide (CHP) that includes one or more binding partners crosslinked to a CHP. The CHP has a sequence represented by Formula I: (Gly-X-Y)_{a-b}, wherein Gly is glycine, at least one of X and Y is proline, modified proline and/or hydroxyproline, and a is 3 and b is 20. The one or more binding partners is selected from the group consisting of integrin sites, integrin binding sites, crosslinking sites, von Willebrand Factor (VWF) discoidin domain receptor (DDR) 1 and 2, SPARC binding peptides, fibronectin binding peptides, engineered integrin binding peptides, matrix metalloproteinase (MMP) cleavage sites, Cathepsin K (CATK) sites, and osteoclast-associated receptors (OSCAR).

IIIa. Collagen Hybridizing Peptides (CHPs)

Collagen is the most abundant protein in a human body and is a critical component of almost all organs and tissues, providing the framework for cell attachment and growth. All types of collagen from all species share the triple helical protein structure, which is nearly exclusively found in collagen.

In certain embodiments, CHPs comprise a short repeating tripeptide that is capable of specifically targeting damaged collagen in the tissues by recognizing a structural motif (e.g., the poly-proline II-like helix of alpha chains) which are not available on intact collagen molecules. By modifying the CHPs to induce specific biological activity, the CHPs can then be used to enhance the microenvironment around the fibroblasts so they can adhere to the ECM and modulate collagen production by increasing mechanical stimulation and feedback within the ECM.

In certain embodiments, the CHPs include specific sequences (also referred to herein as “active sequences or bioactive sequences”) that can crosslink to triple-helical forming sections on both sides of the bioactive sequence. The fibroblasts recognize the bioactive sequences once they are in triple helical form, thereby preventing the fibroblasts from binding the bioactive sequences unless the CHPs target the damaged collagen and form a triple helix, and thus ensure the bioactive sequences are in a triple helical structure. Therefore, the modified CHPs of the present disclosure do not bind fibroblasts in solution. Instead, the binding between the fibroblasts and the modified CHPs only occurs when the fibroblasts have localized to the CHPs which are bound to damaged collagen.

### IV. Modified Collagen Hybridizing Peptides (CHPs)

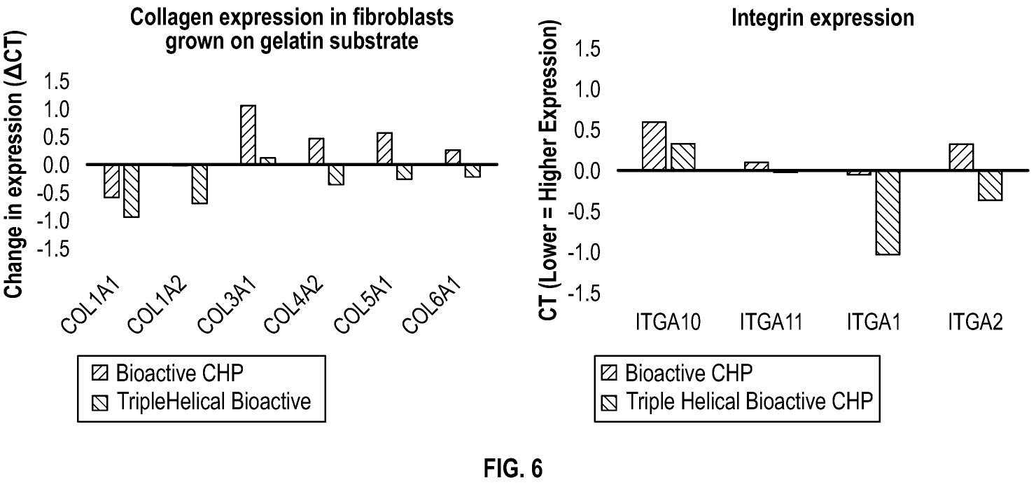

FIG. 6 shows the results from the bioACTIVE CHP with the sequence, CF-Ahx-(GPO)₃-GFOGER-(GPO)₃. The GFOGER is a binding site for the α1β1 and α2β1 integrins. The modified CHP also includes sequences with a high propensity for forming triple helices with GPO repeats on either side of the integrin binding site, GFOGER. These sequences ensure that the CHP can stably bind to the damaged collagen. Thus, in one example, the modified CHP has a sequence CF-Ahx-(GPO)₃-GFOGER-(GPO)₃. The modified CHP, incorporates into the gelatin (coated on the slides) forming collagen triple helix such that the integrin binding site, GFOGER, is only present only on the CHP. α2β1 integrin primarily interacts with only a single alpha chain, with a few interactions occurring on a second alpha chain and none from the third alpha chain of the collagen triple helix. Consequently, binding between the fibroblasts and the integrin binding domains occurs where the CHPs bind to the degraded collagen.

In certain embodiments, the active sequences are selected to be binding sites for molecules and/or markers that are specific to fibroblasts so as to recruit and/or bind fibroblasts at sites where collagen is degraded or damaged, thereby stimulating fibroblasts to either increase collagen production, remove damaged collagen faster, decrease MMP production or some combination of the above to induce tissue repair. Accordingly, the one or more binding partners may include, but are not limited to, an MMP cleavage site, an integrin site, an integrin binding site, a fibronectin binding site, a cathepsin K (CATK) binding site, a crosslinking site, von Willebrand Factor (VWF), discoidin domain receptor (DDR) 1, DDR2, SPARC binding peptide, an engineered integrin binding peptide, and osteoclast-associated receptors (OSCAR).

In certain embodiments, the modified CHP disclosed herein comprises one or more binding site crosslinked to a CHP. The CHP includes a sequence represented by Formula I: (Gly-X-Y) a-b, in which Gly is Glycine, at least one of X and Y is proline, hydroxyproline and/or a modified proline, and a is 3 and b is 20. The one or more binding partners is selected from the group consisting of integrin sites, integrin binding sites, crosslinking sites, von Willebrand Factor (VWF), discoidin domain receptor (DDR) 1, DDR2, SPARC binding peptide, fibronectin binding peptides, engineered integrin binding peptides, matrix metalloproteinase (MMP) cleavage sites, Cathepsin K (CATK) sites, and osteoclast-associated receptors (OSCAR).

In some embodiments, the modified CHP comprises an integrin site crosslinked to a CHP having a sequence represented by Formula I: (Gly-X-Y)_{a-b}, in which Gly is glycine: at least one of X and Y is proline, modified proline, and/or hydroxyproline; and a is 3 and b is 20.

In an exemplary embodiment, a is 3 and b is 4. In an exemplary embodiment, a is 3 and b is 5. In an exemplary embodiment, a is 3 and b is 6. In an exemplary embodiment, a is 3 and b is 7. In an exemplary embodiment, a is 3 and b is 8. In an exemplary embodiment, a is 3 and b is 9. In an exemplary embodiment, a is 3 and b is 10. In an exemplary embodiment, a is 3 and b is 11. In an exemplary embodiment, a is 3 and b is 12. In an exemplary embodiment, a is 3 and b is 13. In an exemplary embodiment, a is 3 and b is 14. In an exemplary embodiment, a is 3 and b is 15. In an exemplary embodiment, a is 3 and b is 16. In an exemplary embodiment, a is 3 and b is 17. In an exemplary embodiment, a is 3 and b is 18. In an exemplary embodiment, a is 3 and b is 19. In an exemplary embodiment, a is 3 and b is 20.

In an exemplary embodiment, a is 4 and b is 5. In an exemplary embodiment, a is 4 and b is 6. In an exemplary embodiment, a is 4 and b is 7. In an exemplary embodiment, a is 4 and b is 8. In an exemplary embodiment, a is 4 and b is 9. In an exemplary embodiment, a is 4 and b is 10. In an exemplary embodiment, a is 4 and b is 11. In an exemplary embodiment, a is 4 and b is 12. In an exemplary embodiment, a is 4 and b is 13. In an exemplary embodiment, a is 4 and b is 14. In an exemplary embodiment, a is 4 and b is 15. In an exemplary embodiment, a is 4 and b is 16. In an exemplary embodiment, a is 4 and b is 17. In an exemplary embodiment, a is 4 and b is 18. In an exemplary embodiment, a is 4 and b is 19. In an exemplary embodiment, a is 4 and b is 20.

In an exemplary embodiment, a is 5 and b is 6. In an exemplary embodiment, a is 5 and b is 7. In an exemplary embodiment, a is 5 and b is 8. In an exemplary embodiment, a is 5 and b is 9. In an exemplary embodiment, a is 5 and b is 10. In an exemplary embodiment, a is 5 and b is 11. In an exemplary embodiment, a is 5 and b is 12. In an exemplary embodiment, a is 5 and b is 13. In an exemplary embodiment, a is 5 and b is 14. In an exemplary embodiment, a is 5 and b is 15. In an exemplary embodiment, a is 5 and b is 16. In an exemplary embodiment, a is 5 and b is 17. In an exemplary embodiment, a is 5 and b is 18. In an exemplary embodiment, a is 5 and b is 19. In an exemplary embodiment, a is 5 and b is 20.

In an exemplary embodiment, a is 6 and b is 7. In an exemplary embodiment, a is 6 and b is 8. In an exemplary embodiment, a is 6 and b is 9. In an exemplary embodiment, a is 6 and b is 10. In an exemplary embodiment, a is 6 and b is 11. In an exemplary embodiment, a is 6 and b is 12. In an exemplary embodiment, a is 6 and b is 13. In an exemplary embodiment, a is 6 and b is 14. In an exemplary embodiment, a is 6 and b is 15. In an exemplary embodiment, a is 6 and b is 16. In an exemplary embodiment, a is 6 and b is 17. In an exemplary embodiment, a is 6 and b is 18. In an exemplary embodiment, a is 6 and b is 19. In an exemplary embodiment, a is 6 and b is 20.

In an exemplary embodiment, a is 7 and b is 8. In an exemplary embodiment, a is 7 and b is 9. In an exemplary embodiment, a is 7 and b is 10. In an exemplary embodiment, a is 7 and b is 11. In an exemplary embodiment, a is 7 and b is 12. In an exemplary embodiment, a is 7 and b is 13. In an exemplary embodiment, a is 7 and b is 14. In an exemplary embodiment, a is 7 and b is 15. In an exemplary embodiment, a is 7 and b is 17. In an exemplary embodiment, a is 7 and b is 17. In an exemplary embodiment, a is 7 and b is 18. In an exemplary embodiment, a is 7 and b is 19. In an exemplary embodiment, a is 7 and b is 20.

In an exemplary embodiment, a is 8 and b is 9. In an exemplary embodiment, a is 8 and b is 10. In an exemplary embodiment, a is 8 and b is 11. In an exemplary embodiment, a is 8 and b is 12. In an exemplary embodiment, a is 8 and b is 13. In an exemplary embodiment, a is 8 and b is 14. In an exemplary embodiment, a is 8 and b is 15. In an exemplary embodiment, a is 8 and b is 16. In an exemplary embodiment, a is 8 and b is 17. In an exemplary embodiment, a is 8 and b is 18. In an exemplary embodiment, a is 8 and b is 19. In an exemplary embodiment, a is 8 and b is 20.

In an exemplary embodiment, a is 9 and b is 10. In an exemplary embodiment, a is 9 and b is 11. In an exemplary embodiment, a is 9 and b is 12. In an exemplary embodiment, a is 9 and bis 13. In an exemplary embodiment, a is 9 and b is 14. In an exemplary embodiment, a is 9 and bis 15. In an exemplary embodiment, a is 9 and b is 16. In an exemplary embodiment, a is 9 and b is 17. In an exemplary embodiment, a is 9 and b is 18. In an exemplary embodiment, a is 9 and b is 19. In an exemplary embodiment, a is 9 and b is 20.

In an exemplary embodiment, a is 10 and b is 11. In an exemplary embodiment, a is 10 and b is 12. In an exemplary embodiment, a is 10 and b is 13. In an exemplary embodiment, a is 10 and b is 14. In an exemplary embodiment, a is 10 and b is 15. In an exemplary embodiment, a is 10 and b is 16. In an exemplary embodiment, a is 10 and b is 17. In an exemplary embodiment, a is 10 and b is 18. In an exemplary embodiment, a is 10 and b is 19. In an exemplary embodiment, a is 10 and b is 20.

In an exemplary embodiment, a is 11 and b is 12. In an exemplary embodiment, a is 11 and b is 13. In an exemplary embodiment, a is 11 and b is 14. In an exemplary embodiment, a is 11 and b is 15. In an exemplary embodiment, a is 11 and b is 16. In an exemplary embodiment, a is 11 and b is 17. In an exemplary embodiment, a is 11 and b is 18. In an exemplary embodiment, a is 11 and b is 19. In an exemplary embodiment, a is 11 and b is 20.

In an exemplary embodiment, a is 12 and b is 13. In an exemplary embodiment, a is 12 and b is 14. In an exemplary embodiment, a is 12 and b is 15. In an exemplary embodiment, a is 12 and b is 16. In an exemplary embodiment, a is 12 and b is 17. In an exemplary embodiment, a is 12 and b is 18. In an exemplary embodiment, a is 12 and b is 19. In an exemplary embodiment, a is 12 and b is 20.

In an exemplary embodiment, a is 13 and b is 14. In an exemplary embodiment, a is 13 and b is 15. In an exemplary embodiment, a is 13 and b is 16. In an exemplary embodiment, a is 13 and b is 17. In an exemplary embodiment, a is 13 and b is 18. In an exemplary embodiment, a is 13 and b is 19. In an exemplary embodiment, a is 13 and b is 20.

In an exemplary embodiment, a is 14 and b is 15. In an exemplary embodiment, a is 14 and b is 16. In an exemplary embodiment, a is 14 and b is 17. In an exemplary embodiment, a is 14 and b is 18. In an exemplary embodiment, a is 14 and b is 19. In an exemplary embodiment, a is 14 and b is 20.

In an exemplary embodiment, a is 15 and b is 16. In an exemplary embodiment, a is 15 and b is 17. In an exemplary embodiment, a is 15 and b is 18. In an exemplary embodiment, a is 15 and b is 19. In an exemplary embodiment, a is 15 and b is 20.

In an exemplary embodiment, a is 16 and b is 17. In an exemplary embodiment, a is 16 and b is 18. In an exemplary embodiment, a is 16 and b is 19. In an exemplary embodiment, a is 16 and b is 20.

In an exemplary embodiment, a is 17 and b is 18. In an exemplary embodiment, a is 17 and b is 19. In an exemplary embodiment, a is 17 and b is 20.

In an exemplary embodiment, a is 18 and b is 19. In an exemplary embodiment, a is 18 and b is 20. In an exemplary embodiment, a is 19 and b is 20.

In some embodiments, the modified CHP comprises an integrin binding site crosslinked to a CHP having a sequence represented by Formula I: (Gly-X-Y)_{a-b}, in which Gly is glycine; at least one of X and Y is proline, modified proline, and/or hydroxyproline; and a is 3 and b is 20.

In some embodiments, the CHP described herein comprises the sequence of any one of SEQ ID NOs: 1-169 shown in Table 1 below.

TABLE 1
Sequences for Collagen Hybridizing Peptides

| | Sequence |
|---|---|
| Identifier | (One-Letter Amino Acid Symbols) |
| SEQ ID NO: 1 | GPOGPOGPOGPOGPOGPOGPOGPOGPO-NH₂ |
| SEQ ID NO: 2 | GPOGPOGPOGPOGPOGPOGPOGPOGPO-NH₂ |
| SEQ ID NO: 3 | GPOGPOGPOGPOGPOGPOGPOGPOGPO-NH₂ |
| SEQ ID NO: 4 | GPOGPOGPOGPOGPOGPOGPOGPOGPO-NH₂ |
| SEQ ID NO: 5 | GPOGPOGPOGPOGPOGPOGPOGPOGPO-NH₂ |
| SEQ ID NO: 6 | GPOGPOGPOGPOGPOGPOGPOGPOGPO-NH₂ |
| SEQ ID NO: 7 | GPOGPOGPOGPOGPOGPOGPOGPOGPO-NH₂ |
| SEQ ID NO: 8 | GPOGPOGPOGPOGPOGPOGPOGPOGPO-NH₂ |
| SEQ ID NO: 9 | GPOGPOGPOGPOGPOGPOGPOGPOGPO-NH₂ |
| SEQ ID NO: 10 | GPOGPOGPOGPOGPOGPOGPOGPOGPO-NH₂ |
| SEQ ID NO: 11 | GPOGPOGPOGPOGPOGPOGPOGPOGPO-NH₂ |
| SEQ ID NO: 12 | GPOGPOGPOGPOGPOGPOGPOGPOGPO-NH₂ |
| SEQ ID NO: 13 | GPOGPOGPOGPOGPOGPOGPOGPOGPO-NH₂ |
| SEQ ID NO: 14 | GPOGPOGPOGPOGPOGPOGPOGPOGPO-NH₂ |
| SEQ ID NO: 15 | GPOGPOGPOGPOGPOGPOGPOGPOGPO-NH₂ |
| SEQ ID NO: 16 | GPOGPOGPOGPOGPOGPOGPOGPOGPO-NH₂ |
| SEQ ID NO: 17 | GPOGPOGPOGPOGPOGPOGPOGPOGPO-NH₂ |
| SEQ ID NO: 18 | GPOGPOGPOGPOGPOGPOGPOGPOGPO-NH₂ |
| SEQ ID NO: 19 | GPOGPOGPOGPOGPOGPOGPOGPOGPO-NH₂ |
| SEQ ID NO: 20 | GPOGPOGPOGPOGPOGPOGPOGPOGPO-NH₂ |
| SEQ ID NO: 21 | GPOGPOGPOGPOGPOGPOGPOGPOGPO-NH₂ |
| SEQ ID NO: 22 | GPOGPOGPOGPOGPOGPOGPOGPOGPO-NH₂ |
| SEQ ID NO: 23 | GPOGPOGPOGPOGPOGPOGPOGPOGPO-NH₂ |
| SEQ ID NO: 24 | GPOGPOGPOGPOGPOGPOGPOGPOGPO-NH₂ |
| SEQ ID NO: 25 | GPOGPOGPOGPOGPOGPOGPOGPOGPO-NH₂ |
| SEQ ID NO: 26 | GPOGPOGPOGPOGPOGPOGPOGPOGPO-NH₂ |
| SEQ ID NO: 27 | GPOGPOGPOGPOGPOGPOGPOGPOGPO-NH₂ |
| SEQ ID NO: 28 | GPOGPOGPOGPOGPOGPOGPOGPOGPO-NH₂ |
| SEQ ID NO: 29 | GfOGfOGfOGfOGfOGfOGfOGfOGfO-NH₂ |
| SEQ ID NO: 30 | GfOGfOGfOGfOGfOGfOGfOGFOGfO-NH₂ |
| SEQ ID NO: 31 | GfOGfOGfOGfOGfOGfOGfOGfOGfO-NH₂ |
| SEQ ID NO: 32 | GfOGfOGfOGfOGfOGfOGfOGfOGfO-NH₂ |
| SEQ ID NO: 33 | GfOGfOGfOGfOGfOGfOGfOGfOGfO-NH₂ |
| SEQ ID NO: 34 | GfOGfOGfOGfOGfOGfOGfOGfOGfO-NH₂ |
| SEQ ID NO: 35 | GfOGfOGfOGfOGfOGfOGfOGfOGfO-NH₂ |
| SEQ ID NO: 36 | GfOGfOGfOGfOGfOGfOGfOGfOGfO-NH₂ |
| SEQ ID NO: 37 | GfOGfOGfOGfOGfOGfOGfOGfOGfO-NH₂ |
| SEQ ID NO: 38 | GfOGfOGfOGfOGfOGfOGfOGfOGfO-NH₂ |
| SEQ ID NO: 39 | GfOGfOGfOGfOGfOGfOGfOGfOGfO-NH₂ |
| SEQ ID NO: 40 | GfOGfOGfOGfOGfOGfOGfOGfOGfO-NH₂ |
| SEQ ID NO: 41 | GfOGfOGfOGfOGfOGfOGfOGfOGfO-NH₂ |
| SEQ ID NO: 42 | GfOGfOGfOGfOGfOGfOGfOGfOGfO-NH₂ |
| SEQ ID NO: 43 | GfOGfOGfOGfOGfOGfOGfOGfOGfO-NH₂ |
| SEQ ID NO: 44 | GfOGfOGfOGfOGfOGfOGfOGfOGfO-NH₂ |
| SEQ ID NO: 45 | GfOGfOGfOGfOGfOGfOGfOGfOGfO-NH₂ |
| SEQ ID NO: 46 | GfOGfOGfOGfOGfOGfOGfOGfOGfO-NH₂ |
| SEQ ID NO: 47 | GfOGfOGfOGfOGfOGfOGfOGfOGfO-NH₂ |
| SEQ ID NO: 48 | GfOGfOGfOGfOGfOGfOGfOGfOGfO-NH₂ |
| SEQ ID NO: 49 | GfOGfOGfOGfOGfOGfOGfOGfOGfO-NH₂ |
| SEQ ID NO: 50 | GfOGfOGfOGfOGfOGfOGfOGfOGfO-NH₂ |
| SEQ ID NO: 51 | GfOGfOGfOGfOGfOGfOGfOGfOGfO-NH₂ |
| SEQ ID NO: 52 | GfOGfOGfOGfOGfOGfOGfOGfOGfO-NH₂ |
| SEQ ID NO: 53 | GfOGfOGfOGfOGfOGfOGfOGfOGfO-NH₂ |
| SEQ ID NO: 54 | GfOGfOGfOGfOGfOGfOGfOGfOGfO-NH₂ |
| SEQ ID NO: 55 | GfOGfOGfOGfOGfOGfOGfOGfOGfO-NH₂ |
| SEQ ID NO: 56 | GfOGfOGfOGfOGfOGfOGfOGfOGfO-NH₂ |
| SEQ ID NO: 57 | GPOGPOGPOGPOGPOGPOGPOGPOGPO-NH₂ |
| SEQ ID NO: 58 | GPOGPOGPOGPOGPOGPOGPOGPOGPO-NH₂ |
| SEQ ID NO: 59 | GPOGPOGPOGPOGPOGPOGPOGPOGPO-NH₂ |
| SEQ ID NO: 60 | GPOGPOGPOGPOGPOGPOGPOGPOGPO-NH₂ |
| SEQ ID NO: 61 | GPOGPOGPOGPOGPOGPOGPOGPOGPO-NH₂ |
| SEQ ID NO: 62 | GPOGPOGPOGPOGPOGPOGPOGPOGPO-NH₂ |
| SEQ ID NO: 63 | GPOGPOGPOGPOGPOGPOGPOGPOGPO-NH₂ |
| SEQ ID NO: 64 | GPOGPOGPOGPOGPOGPOGPOGPOGPO-NH₂ |
| SEQ ID NO: 65 | GPOGPOGPOGPOGPOGPOGPOGPOGPO-NH₂ |
| SEQ ID NO: 66 | GPOGPOGPOGPOGPOGPOGPOGPOGPO-NH₂ |
| SEQ ID NO: 67 | GPOGPOGPOGPOGPOGPOGPOGPOGPO-NH₂ |
| SEQ ID NO: 68 | GPOGPOGPOGPOGPOGPOGPOGPOGPO-NH₂ |
| SEQ ID NO: 69 | GPOGPOGPOGPOGPOGPOGPOGPOGPO-NH₂ |
| SEQ ID NO: 70 | GPOGPOGPOGPOGPOGPOGPOGPOGPO-NH₂ |
| SEQ ID NO: 71 | GPOGPOGPOGPOGPOGPOGPOGPOGPO-NH₂ |
| SEQ ID NO: 72 | GPOGPOGPOGPOGPOGPOGPOGPOGPO-NH₂ |
| SEQ ID NO: 73 | GPOGPOGPOGPOGPOGPOGPOGPOGPO-NH₂ |
| SEQ ID NO: 74 | GPOGPOGPOGPOGPOGPOGPOGPOGPO-NH₂ |
| SEQ ID NO: 75 | GPOGPOGPOGPOGPOGPOGPOGPOGPO-NH₂ |
| SEQ ID NO: 76 | GPOGPOGPOGPOGPOGPOGPOGPOGPO-NH₂ |
| SEQ ID NO: 77 | GPOGPOGPOGPOGPOGPOGPOGPOGPO-NH₂ |
| SEQ ID NO: 78 | GPOGPOGPOGPOGPOGPOGPOGPOGPO-NH₂ |
| SEQ ID NO: 79 | GPOGPOGPOGPOGPOGPOGPOGPOGPO-NH₂ |
| SEQ ID NO: 80 | GPOGPOGPOGPOGPOGPOGPOGPOGPO-NH₂ |
| SEQ ID NO: 81 | GPOGPOGPOGPOGPOGPOGPOGPOGPO-NH₂ |
| SEQ ID NO: 82 | GPOGPOGPOGPOGPOGPOGPOGPOGPO-NH₂ |
| SEQ ID NO: 83 | GPOGPOGPOGPOGPOGPOGPOGPOGPO-NH₂ |
| SEQ ID NO: 84 | GPOGPOGPOGPOGPOGPOGPOGPOGPO-NH₂ |
| SEQ ID NO: 85 | GfOGfOGfOGfOGfOGfOGfOGfOGfO-NH₂ |
| SEQ ID NO: 86 | GfOGfOGfOGfOGfOGfOGfOGfOGfO-NH₂ |
| SEQ ID NO: 87 | GfOGfOGfOGfOGfOGfOGfOGfOGfO-NH₂ |
| SEQ ID NO: 88 | GfOGfOGfOGfOGfOGfOGfOGfOGfO-NH₂ |
| SEQ ID NO: 89 | GfOGfOGfOGfOGfOGfOGfOGfOGfO-NH₂ |
| SEQ ID NO: 90 | GfOGfOGfOGfOGfOGfOGfOGfOGfO-NH₂ |
| SEQ ID NO: 91 | GfOGfOGfOGfOGfOGfOGfOGfOGfO-NH₂ |
| SEQ ID NO: 92 | GfOGfOGfOGfOGfOGfOGfOGfOGfO-NH₂ |
| SEQ ID NO: 93 | GfOGfOGfOGfOGfOGfOGfOGfOGfO-NH₂ |
| SEQ ID NO: 94 | GfOGfOGfOGfOGfOGfOGfOGfOGfO-NH₂ |
| SEQ ID NO: 95 | GfOGfOGfOGfOGfOGfOGfOGfOGfO-NH₂ |
| SEQ ID NO: 96 | GfOGfOGfOGfOGfOGfOGfOGfOGfO-NH₂ |
| SEQ ID NO: 97 | GfOGfOGfOGfOGfOGfOGfOGfOGfO-NH₂ |
| SEQ ID NO: 98 | GfOGfOGfOGfOGfOGfOGfOGfOGfO-NH₂ |
| SEQ ID NO: 99 | GfOGfOGfOGfOGfOGfOGfOGfOGfO-NH₂ |
| SEQ ID NO: 100 | GfOGfOGfOGfOGfOGfOGfOGfOGfO-NH₂ |
| SEQ ID NO: 101 | GfOGfOGfOGfOGfOGfOGfOGfOGfO-NH₂ |
| SEQ ID NO: 102 | GfOGfOGfOGfOGfOGfOGfOGFOGfO-NH₂ |
| SEQ ID NO: 103 | GfOGfOGfOGfOGfOGfOGfOGfOGfO-NH₂ |
| SEQ ID NO: 104 | GfOGfOGfOGfOGfOGfOGfOGfOGfO-NH₂ |
| SEQ ID NO: 105 | GfOGfOGfOGfOGfOGfOGfOGfOGfO-NH₂ |
| SEQ ID NO: 106 | GfOGfOGfOGfOGfOGfOGfOGfOGfO-NH₂ |
| SEQ ID NO: 107 | GfOGfOGfOGfOGfOGfOGfOGfOGfO-NH₂ |
| SEQ ID NO: 108 | GfOGfOGfOGfOGfOGfOGfOGfOGfO-NH₂ |
| SEQ ID NO: 109 | GfOGfOGfOGfOGfOGfOGfOGfOGfO-NH₂ |
| SEQ ID NO: 110 | GfOGfOGfOGfOGfOGfOGfOGfOGfO-NH₂ |
| SEQ ID NO: 111 | GfOGfOGfOGfOGfOGfOGfOGfOGfO-NH₂ |
| SEQ ID NO: 112 | GfOGfOGfOGfOGfOGfOGfOGfOGfO-NH₂ |
| SEQ ID NO: 113 | GcOGcOGcOGcOGcOGcOGcOGcOGcO-NH₂ |
| SEQ ID NO: 114 | GcOGcOGcOGcOGcOGcOGcOGcOGcO-NH₂ |
| SEQ ID NO: 115 | GcOGcOGcOGcOGcOGcOGcOGcOGcO-NH₂ |
| SEQ ID NO: 116 | GcOGcOGcOGcOGcOGcOGcOGcOGcO-NH₂ |
| SEQ ID NO: 117 | GcOGcOGcOGcOGcOGcOGcOGcOGcO-NH₂ |
| SEQ ID NO: 118 | GcOGcOGcOGcOGcOGcOGcOGcOGcO-NH₂ |
| SEQ ID NO: 119 | GcOGcOGcOGcOGcOGcOGcOGcOGcO-NH₂ |
| SEQ ID NO: 120 | GcOGcOGcOGcOGcOGcOGcOGcOGcO-NH₂ |
| SEQ ID NO: 121 | GcOGcOGcOGcOGcOGcOGcOGcOGcO-NH₂ |
| SEQ ID NO: 122 | GcOGcOGcOGcOGcOGcOGcOGcOGcO-NH₂ |
| SEQ ID NO: 123 | GcOGcOGcOGcOGcOGcOGcOGcOGcO-NH₂ |
| SEQ ID NO: 124 | GcOGcOGcOGcOGcOGcOGcOGcOGcO-NH₂ |
| SEQ ID NO: 125 | GcOGcOGcOGcOGcOGcOGcOGcOGcO-NH₂ |
| SEQ ID NO: 126 | GcOGcOGcOGcOGcOGcOGcOGcOGcO-NH₂ |
| SEQ ID NO: 127 | GcOGcOGcOGcOGcOGcOGcOGcOGcO-NH₂ |
| SEQ ID NO: 128 | GcOGcOGcOGcOGcOGcOGcOGcOGcO-NH₂ |
| SEQ ID NO: 129 | GcOGcOGcOGcOGcOGcOGcOGcOGcO-NH₂ |
| SEQ ID NO: 130 | GcOGcOGcOGcOGcOGcOGcOGcOGCO-NH₂ |
| SEQ ID NO: 131 | GcOGcOGcOGcOGcOGcOGcOGcOGcO-NH₂ |
| SEQ ID NO: 132 | GcOGcOGcOGcOGcOGcOGcOGcOGcO-NH₂ |
| SEQ ID NO: 134 | GcOGcOGcOGcOGcOGcOGcOGcOGcO-NH₂ |
| SEQ ID NO: 135 | GcOGcOGcOGcOGcOGcOGcOGcOGcO-NH₂ |
| SEQ ID NO: 136 | GcOGcOGcOGcOGcOGcOGcOGcOGcO-NH₂ |
| SEQ ID NO: 137 | GcOGcOGcOGcOGcOGcOGcOGcOGcO-NH₂ |
| SEQ ID NO: 138 | GcOGcOGcOGcOGcOGcOGcOGcOGcO-NH₂ |
| SEQ ID NO: 139 | GcOGcOGcOGcOGcOGcOGcOGcOGcO-NH₂ |
| SEQ ID NO: 140 | GcOGcOGcOGcOGcOGcOGcOGcOGcO-NH₂ |
| SEQ ID NO: 141 | GcOGcOGcOGcOGcOGcOGcOGcOGcO-NH₂ |
| SEQ ID NO: 142 | GcOGcOGcOGcOGcOGcOGcOGcOGcO-NH₂ |
| SEQ ID NO: 143 | GcOGcOGcOGcOGcOGcOGcOGcOGcO-NH₂ |
| SEQ ID NO: 144 | GcOGcOGcOGcOGcOGcOGcOGcOGcO-NH₂ |
| SEQ ID NO: 145 | GcOGcOGcOGcOGcOGcOGcOGcOGcO-NH₂ |
| SEQ ID NO: 146 | GcOGcOGcOGcOGcOGcOGcOGcOGcO-NH₂ |
| SEQ ID NO: 147 | GcOGcOGcOGcOGcOGcOGcOGcOGcO-NH₂ |
| SEQ ID NO: 148 | GcOGcOGcOGcOGcOGcOGcOGcOGcO-NH₂ |
| SEQ ID NO: 149 | GcOGcOGcOGcOGcOGcOGcOGcOGcO-NH₂ |
| SEQ ID NO: 150 | GcOGcOGcOGcOGcOGcOGcOGcOGcO-NH₂ |
| SEQ ID NO: 151 | GcOGcOGcOGcOGcOGcOGcOGcOGcO-NH₂ |
| SEQ ID NO: 152 | GcOGcOGcOGcOGcOGcOGcOGcOGcO-NH₂ |
| SEQ ID NO: 153 | GcOGcOGcOGcOGcOGcOGcOGcOGcO-NH₂ |
| SEQ ID NO: 154 | GcOGcOGcOGcOGcOGcOGcOGcOGcO-NH₂ |
| SEQ ID NO: 155 | GcOGcOGcOGcOGcOGcOGcOGcOGcO-NH₂ |
| SEQ ID NO: 156 | GcOGcOGcOGcOGcOGcOGcOGcOGcO-NH₂ |
| SEQ ID NO: 157 | GcOGcOGcOGcOGcOGcOGcOGcOGcO-NH₂ |
| SEQ ID NO: 158 | GcOGcOGcOGcOGcOGcOGcOGcOGcO-NH₂ |
| SEQ ID NO: 159 | GcOGcOGcOGcOGcOGcOGcOGcOGcO-NH₂ |
| SEQ ID NO: 160 | GcOGcOGcOGcOGcOGcOGcOGcOGcO-NH₂ |
| SEQ ID NO: 161 | GcOGcOGcOGcOGcOGcOGcOGcOGcO-NH₂ |
| SEQ ID NO: 162 | GcOGcOGcOGcOGcOGcOGcOGcOGcO-NH₂ |
| SEQ ID NO: 163 | GcOGcOGcOGcOGcOGcOGcOGcOGcO-NH₂ |
| SEQ ID NO: 164 | GcOGcOGcOGcOGcOGcOGcOGcOGcO-NH₂ |
| SEQ ID NO: 165 | GcOGcOGcOGcOGcOGcOGcOGcOGcO-NH₂ |
| SEQ ID NO: 166 | GcOGcOGcOGcOGcOGcOGcOGcOGCO-NH₂ |
| SEQ ID NO: 167 | GcOGcOGcOGcOGcOGcOGcOGcOGcO-NH₂ |
| SEQ ID NO: 168 | GcOGcOGcOGcOGcOGcOGcOGcOGcO-NH₂ |
| SEQ ID NO: 169 | GcOGcOGcOGcOGcOGcOGcOGcOGcO-NH₂ |

In certain sequences provided in Table 1 above, ‘NH₂’ represents an amidated C-terminus. In certain sequences provided in Table 1 above, the ‘f’ in a ‘GfO’ sequence represents a 2S, 4S-4-fluoroproline (cis conformation). In certain sequences provided in Table 1 above, the “c” in a ‘GcO’ sequence represents a c=cis-chloroproline (2S,4S-4-chloroproline).

In some embodiments, the CHP has a sequence selected from Table 2 below.

TABLE 2
Sequences for Repeating Portion of Collagen Hybridizing Peptides
Sequence (One-Letter Amino Acid Symbols)
(GPO)₃₋₂₀, where G = glycine, P = proline, O = trans-hydroxyproline
(2S, 4R-hydroxyproline)
(GfO)₃₋₂₀, where G = glycine, f = cis-fluoroproline (2S,4S-4-
fluoroproline), O = trans-hydroxyproline (2S, 4R-
hydroxyproline)
(GcO)₃₋₂₀, where G = glycine, c = cis-chloroproline (2S,4S-4-
chloroproline), O = trans-hydroxyproline (2S, 4R-
hydroxyproline)
(GOP)₃₋₂₀, where G = glycine, P = proline, O = trans-hydroxyproline
(2S, 4R-hydroxyproline)
(_{az}GPO)₃₋₂₀, where _{az}G = aza-glycine, P = proline, O = trans-
hydroxyproline (2S, 4R-hydroxyproline)
(_{az}GfO)₃₋₂₀, where _{az}G = aza-glycine, f = cis-fluoroproline (2S,4S-4-
fluoroproline), O = trans-hydroxyproline (2S, 4R-
hydroxyproline)
(GPP)₃₋₂₀, where G = glycine, P = proline, P = proline
(_{az}GPP)₃₋₂₀, where _{az}G = aza-glycine, P = proline, P = proline
(_{az}GcO)₃₋₂₀, where _{az}G = aza-glycine, c = cis-chloroproline (2S,4S-4-
chloroproline), O = trans-hydroxyproline (2S, 4R-
hydroxyproline)

In some embodiments, the modified CHP comprises an integrin binding site crosslinked to a CHP having a sequence represented by Formula I: (Gly-X-Y)_{a-b}, in which Gly is glycine; at least one of X and Y is proline, modified proline, and/or hydroxyproline; and a is 3 and b is 20. The integrin binding site includes an integrin, α1β1, and has a sequence selected the sequences listed in Table 3:

TABLE 3
Modified CHP sequences having integrin α1ß1 binding partner.

| SEQ | | | |
|---|---|---|---|
| ID. | Site | | |
| NO. | Name | Potential CHP Sequence (1) | Potential CHP Sequence (2) |
| 170 | α1ß1 | GPPGPPGPPGPPGPPGFPGERGPPGPPGPP | GfOGfOGfOGfOGfOGFPGERGfOGfOGfO |
| | | GPPGPP | GfOGfO |
| 171 | α1ß1 | GPPGPPGPPGPPGPPGLOGERGPPGPPGPP | GfOGfOGfOGfOGfOGLOGERGfOGfOGfO |
| | | GPPGPP | GfOGfO |
| 172 | α2ß1, | GPPGPPGPPGPPGPPGROGERGPPGPPGPP | GfOGfOGfOGfOGfOGROGERGfOGfOGfO |
| | α1ß1 | GPPGPP | GfOGfO |
| 173 | α2ß1, | GPPGPPGPPGPPGPPGAOGERGPPGPPGPP | GfOGfOGfOGfOGfOGAOGERGfOGfOGfO |
| | α1ß1 | GPPGPP | GfOGfO |
| 174 | α1ß1 | GPPGPPGPPGPPGPPGFOGERGPPGPPGPP | GfOGfOGfOGfOGfOGFOGERGfOGfOGfO |
| | | GPPGPP | GfOGfO |
| 175 | α1ß1 | GPPGPPGPPGPPGPPGLOGENGPPGPPGPP | GfOGfOGfOGfOGfOGLOGENGfOGfOGfO |
| | | GPPGPP | GfOGfO |
| 176 | α1ß1 | GPPGPPGPPGPPGPPGVOGEAGPPGPPGPP | GfOGfOGfOGfOGfOGVOGEAGfOGfOGfO |
| | | GPPGPP | GfOGfO |
| 177 | α1ß1, | GPPGPPGPPGPPGPPGFPGENGPPGPPGPP | GfOGfOGfOGfOGfOGFPGENGfOGfOGfO |
| | α10ß1, | GPPGPP | GfOGfO |
| | and | | |
| | α11ß1 | | |
| 178 | α1ß1, | GPPGPPGPPGPPGPPGFPGERGPPGPPGPP | GfOGfOGfOGfOGfOGFPGERGfOGfOGfO |
| | α10ß1, | GPPGPP | GfOGfO |
| | and | | |
| | α11ß1 | | |
| 179 | α2ß1, | GPPGPPGPPGPPGPPGLOGERGROGPPGPP | GfOGfOGfOGfOGfOGLOGERGROGfOGf |
| | α1ß1 | GPPGPPGPP | OGfOGfOGfO |

In some embodiments, the modified CHP comprises an integrin binding site crosslinked to a CHP having a sequence represented by Formula I: (Gly-X-Y)_{a-b}, in which Gly is glycine; at least one of X and Y is proline, modified proline, and/or hydroxyproline; and a is 3 and b is 20. The integrin binding site includes an integrin α2β1, and has a sequence selected the sequences listed in Table 4:

TABLE 4
Modified CHP sequences having integrin α2ß1 binding partner.

| SEQ | | | |
|---|---|---|---|
| ID | Site | | |
| NO. | Name | Potential CHP Sequence (1) | Potential CHP Sequence (2) |
| 180 | α2ß1 | GPPGPPGPPGPPGPPGFOGERGPPGPPGPP | GfOGfOGfOGfOGfOGFOGERGfOGfOGfO |
| | | GPPGPP | GfOGfO |
| 181 | α2ß1, | GPPGPPGPPGPPGPPGROGERGPPGPPGPP | GfOGfOGfOGfOGfOGROGERGfOGfOGfO |
| | α1ß1 | GPPGPP | GfOGfO |
| 182 | α2ß1, | GPPGPPGPPGPPGPPGAOGERGPPGPPGP | GfOGfOGfOGfOGfOGAOGERGfOGfOGf |
| | α1ß1 | PGPPGPP | OGfOGfO |
| 183 | α2ß1, | GPPGPPGPPGPPGPPGLOGERGROGPPGP | GfOGfOGfOGfOGfOGLOGERGROGfOGf |
| | α1ß1 | PGPPGPPGPP | OGfOGfOGfO |

In some embodiments, the modified CHP comprises an integrin binding site crosslinked to a CHP having a sequence represented by Formula I: (Gly-X-Y)_{a-b}, in which Gly is glycine; at least one of X and Y is proline, modified proline, and/or hydroxyproline; and a is 3 and b is 20. The integrin binding site includes an integrin α10β1, and has a sequence selected the sequences listed in Table 5:

TABLE 5
Modified CHP sequences having integrin α10ß1 binding partner.

| SEQ | | | |
|---|---|---|---|
| ID. | Site | | |
| NO. | Name | Potential CHP Sequence (1) | Potential CHP Sequence (2) |
| 184 | α10ß1 | GPPGPPGPPGPPGPPGLOGERGPPGPPGPP | GfOGfOGfOGfOGfOGLOGERGfOGfOGfO |
| | | GPPGPP | GfOGfO |
| 185 | α11ß1 | GPPGPPGPPGPPGPPGFOGERGPPGPPGPP | GfOGfOGfOGfOGfOGFOGERGfOGfOGfO |
| | | GPPGPP | GfOGfO |
| 186 | α1ß1, | GPPGPPGPPGPPGPPGFPGENGPPGPPGPP | GfOGfOGfOGfOGfOGFPGENGfOGfOGfO |
| | α10ß1, | GPPGPP | GfOGfO |
| | and | | |
| | α11ß1 | | |
| 187 | α1ß1, | GPPGPPGPPGPPGPPGFPGERGPPGPPGPP | GfOGfOGfOGfOGfOGFPGERGfOGfOGfO |
| | α10ß1, | GPPGPP | GfOGfO |
| | and | | |
| | α11ß1 | | |

In some embodiments, the modified CHP comprises an integrin binding site crosslinked to a CHP having a sequence represented by Formula I: (Gly-X-Y)_{a-b}, in which Gly is glycine; at least one of X and Y is proline, modified proline, and/or hydroxyproline; and a is 3 and b is 20. The integrin binding site includes an integrin α11β1, and has a sequence selected the sequences listed in Table 6:

TABLE 6
Modified CHP sequences having integrin α11ß1 binding partner.

| SEQ | | | |
|---|---|---|---|
| ID | Site | | |
| NO. | Name | Potential CHP Sequence (1) | Potential CHP Sequence (2) |
| 188 | α11ß1 | GPPGPPGPPGPPGPPGFOGERGPPGPPGP | GfOGfOGfOGfOGfOGFOGERGfOGfOGf |
| | | PGPPGPP | OGfOGfO |
| 189 | α1ß1, | GPPGPPGPPGPPGPPGFPGENGPPGPPGP | GfOGfOGfOGfOGfOGFPGENGfOGfOGf |
| | α10ß1, | PGPPGPP | OGfOGfO |
| | and α11ß1 | | |
| 190 | α1ß1, | GPPGPPGPPGPPGPPGFPGERGPPGPPGP | GfOGfOGfOGfOGfOGFPGERGfOGfOGf |
| | α10ß1, | PGPPGPP | OGfOGfO |
| | and α11ß1 | | |

In some embodiments, the modified CHP comprises crosslinking site crosslinked to a CHP having a sequence represented by Formula I: (Gly-X-Y)a-b, in which Gly is glycine; at least one of X and Y is proline, modified proline, and/or hydroxyproline; and a is 3 and b is 20. The crosslinking site has a sequence selected the sequences listed in Table 7:

TABLE 7
Modified CHP sequences having crosslinking site as a binding partner.

| SEQ | | | |
|---|---|---|---|
| ID | Site | | |
| NO. | Name | Potential CHP Sequence (1) | Potential CHP Sequence (2) |
| 191 | Cross- | GPPGPPGPPGPPGPPGxKGHRGPPGPPGP | GfOGfOGfOGfOGfOGxKGHRGfOGfOGf |
| | linking site | PGPPGPP | OGfOGfO |

In Table 6, “x” refers to an amino acid residue, and in particular a naturally occurring amino acid residue.

In some embodiments, the crosslinking site is a sugar crosslinking site.

In some embodiments, the one or more binding partners includes a crosslinking site including a sequence having at least 85% sequence identity to SEQ ID NO. 17.

In some embodiments, the modified CHP comprises VWF, DDR 1, DDR 2, and SPARC binding peptide crosslinked to a CHP having a sequence represented by Formula I: (Gly-X-Y)a-b, in which Gly is glycine; at least one of X and Y is proline, modified proline, and/or hydroxyproline; and a is 3 and b is 20. The crosslinking site has a sequence selected the sequences listed in Table 8:

TABLE 8
Modified CHP sequences having VWF, DDR 1, DDR 2, and SPARC binding peptide as
a binding partner.

| SEQ | | | |
|---|---|---|---|
| ID | Site | | |
| NO. | Name | Potential CHP Sequence (1) | Potential CHP Sequence (2) |
| 192 | VWFA3 | GPPGPPGPPGPPGPPGxRHQOGVMGFOGP | GfOGfOGfOGfOGfOGxRHOOGVMGFOGf |
| | | PGPPGPPGPPGPP | OGfOGfOGfOGfO |

In Table 8, “x” refers to an amino acid residue, and in particular a naturally occurring amino acid residue.

In some embodiments, the one or more binding partners includes a VWF, DDR 1, DDR 2, and SPARC binding peptide including a sequence having at least 85% sequence identity to SEQ ID NO: 18-21.

In some embodiments, the modified CHP comprises a fibronectin binding motif crosslinked to a CHP having a sequence represented by Formula I: (Gly-X-Y)a-b, in which Gly is glycine; at least one of X and Y is proline, modified proline, and/or hydroxyproline; and a is 3 and b is 20. The crosslinking site has a sequence selected the sequences listed in Table 9:

TABLE 9
Modified CHP sequences having a fibronectin binding motif as a binding partner.

| SEQ | | | |
|---|---|---|---|
| ID | Site | | |
| NO. | Name | Potential CHP Sequence (1) | Potential CHP Sequence (2) |
| 193 | 8-9 Fn | GPPGPPGPPGPPGPPGLOGQRGERGPPG | GfOGfOGfOGfOGfOGLOGQRGERGfOGf |
| | Binding | PPGPPGPPGPP | OGfOGfOGfO |
| 194 | 8-9 Fn | GPPGPPGPPGPPGPPGLOGMKGHRGPPG | GfOGfOGfOGfOGfOGLOGMKGHRGfOG |
| | Binding | PPGPPGPPGPP | fOGfOGfOGfO |

In some embodiments, the one or more binding partners includes a fibronectin binding motif including a sequence having at least 85% sequence identity to SEQ ID NO: 22 or 23.

In some embodiments, the modified CHP comprises an engineered integrin binding motif crosslinked to a CHP having a sequence represented by Formula I: (Gly-X-Y)a-b, in which Gly is glycine; at least one of X and Y is proline, modified proline, and/or hydroxyproline; and a is 3 and b is 20.

In some embodiments, the one or more binding partners includes an engineered integrin binding motif including a sequence having at least 85% sequence identity to SEQ ID NO: 24 or 25.

In some embodiments, the modified CHP comprises an MMP cleavage site crosslinked to a CHP having a sequence represented by Formula I: (Gly-X-Y)a-b, in which Gly is glycine; at least one of X and Y is proline, modified proline, and/or hydroxyproline; and a is 3 and b is 20. The crosslinking site has a sequence selected the sequences listed in Table 10:

TABLE 10
Modified CHP sequences having an MMP cleavage site as a binding partner.

| SEQ | | | |
|---|---|---|---|
| ID | Site | | |
| NO. | Name | Potential CHP Sequence (1) | Potential CHP Sequence (2) |
| 195 | MMP-1 | GPPGPPGPPGPPGPPGPQGIAG(Q)GPPGP | GfOGfOGfOGfOGfOGPQGIAG(Q)GfOGf |
| | and MMP- | PGPPGPPGPP | OGfOGfOGfO |
| | 2 | | |
| 196 | MMP-1 | GPPGPPGPPGPPGPPGPQGIWG(Q)GPPG | GfOGfOGfOGfOGfOGPQGIWG(Q)GfOGf |
| | and MMP- | PPGPPGPPGPP | OGfOGfOGfO |
| | 3 | | |
| 197 | MMP site | GPPGPPGPPGPPGPPGPLGIAGITGARGL | GfOGfOGfOGfOGfOGPLGIAGITGARGL |
| | | AGPPGPPGPPGPPGPP | AGfOGfOGfOGfOGfO |

In some embodiments, the one or more binding partners includes an MMP cleavage site including a sequence having at least 85% sequence identity to SEQ ID NO: 26 or 27.

In some embodiments, the CHP has a sequence selected from the group consisting of SEQ ID NO: XXX.

In some embodiments, the modified CHP has one binding partner, which is crosslinked to C-terminus of the CHP. In some embodiments, the modified CHP has one binding partner, which is crosslinked to N-terminus of the CHP.

In some embodiments, the modified CHP has two binding partners. First of the two binding partners is crosslinked to C-terminus of the CHP and second of the two binding partners is crosslinked to N-terminus of the CHP.

In some embodiments, the one or more binding partners is attached to the C-terminus of the CHP and the N-terminus of the CHP.

In some embodiments, the modified CHP and one or more binding partners are crosslinked directly to the CHP. In some embodiments, the one or more binding partners is crosslinked to the CHP by zero or more spacer. Examples of spacer include, but are not limited to amino acid(s), such as GGG, and chemical spacer(s), such as 6-aminohexanoic acid (Ahx).

In some embodiments, the modified CHP includes a cap at the N-terminus of the CHP. In some embodiments, the cap includes an acetyl group. In some embodiments, the cap is directly attached to the N-terminus of the CHP. In some embodiments, the modified CHP includes a spacer between the N-terminus of the CHP and the cap.

In some embodiments, the one or more binding partners of the modified CHP are attached directly to the N-terminus of the CHP such that the one or more binding partners are attached between the cap and the N-terminus of the CHP. In some embodiments, the modified CHP includes one or more binding partners attached to the N-terminus of the CHP, one or more spacers attached to the one or more binding partners and a cap attached to the one or more spacers such that the one or more spacers are between the cap and the one or more binding partners. In some embodiments, the modified CHP has a structure: cap-spacer-CHP-binding partner-CHP. In some embodiments, the cap and CHP are directly attached. In some embodiments, the cap and the one or more binding partners are attached directly.

### V. Compositions Including Modified CHPs.

In an aspect of the present disclosure, any of the modified CHPs described herein may be included into a therapeutic, medical device, therapeutic medical device, or a cosmetic composition. In some embodiments, the composition is formulated such that the modified CHP included in the composition does not form a triple helix with other modified CHPs.

In some embodiments, the composition includes a carrier, e.g., for carrying the modified CHP across the epidermal layer. Examples of carriers include, but are not limited to, micelles, dendrites, lipids, microemulsions, nanoemulsions, solid lipid nanoparticles, nanostructured lipid carriers, liposomes, transfersomes, ethosomes, niosomes, collagens, extracellular matrix, and artificial extracellular matrix.

In some embodiments, the composition is a cosmetic composition formulated to be administered by an injection or a topical application. In some embodiments, the cosmetic composition may be formulated to be administered via a micro-dermal injection such as, for example, using an array of microneedles including the cosmetic composition.

In some embodiments, compositions including modified CHPs are formulated for administration by local injection, topical application, or physical application.

In some embodiments, the compositions including modified CHPs are formulated as a putty, a mesh, a patch, an adhesive, a cream, sutures, eyedrops, and/or other formulations that can be administered topically or by physical application.

In some embodiments, the compositions including modified CHPs are formulated as a systemic administration through, injection, oral administration, drops, spray, intravenous, intramuscular injection, suppository, enema, vaporizer.

### VI. Uses of Modified CHPs and Compositions Comprising Modified CHPs

As has been discussed herein, the modified CHPs and/or the compositions comprising modified CHPs described herein can be used for modulating fibroblast morphology and inducing fibroblast adherence, thereby inducing fibroblasts to produce more collagen.

Accordingly, the modified CHPs and/or the compositions comprising modified CHPs as described herein can be used for modulating the fibroblast production of collagen or MMPs in a subject. In one aspect increased collagen production results in the decreased appearance of wrinkles and thereby reduces the effects of aging on the skin of the subject. Thus, in an aspect of the present disclosure, a method of increasing collagen production in a subject may include administering the modified CHPs and/or compositions comprising modified CHPs as described herein to the subject. The administration of the modified CHPs and/or compositions comprising modified CHPs may be performed by an injection, a micro-dermal injection or topical application.

Changing the morphology of fibroblasts and inducing the fibroblasts to produce more collagen further reduces the fibroblast-induced production of MMPs, which can augment dermal repair. Accordingly, in an aspect of the present disclosure, a method for decreasing MMP production in a subject may include administering the modified CHPs and/or compositions comprising modified CHPs as described herein to the subject. The administration of the modified CHPs and/or compositions comprising modified CHPs may be performed by an injection, a micro-dermal injection or topical application.

In another aspect, by applying an MMP site as the bioactive site in the CHP, we can increase the collagen degradation rate. Removing the partially damaged collagen faster and more efficiently allowing newly produced collagen to bind to the existing fibrils faster. Thus, by removing damaged collagen, we can effectively restart the collagen fibril formation process, ultimately leading to a higher density and aligned collagen matrix.

Increased collagen production in fibroblasts may support dermal repair in skin that has been damaged by, e.g., trauma or aging. Thus, the modified CHPs and/or the compositions comprising modified CHPs as described herein can be used for inducing dermal repair in a subject in need thereof. Accordingly, in an aspect of the present disclosure, a method for inducing dermal repair in a subject in need thereof may include administering the modified CHPs and/or compositions comprising modified CHPs as described herein to the subject. The compositions comprising the modified CHPs may be cosmetic compositions or therapeutic compositions. In some embodiments, the administration of the modified CHPs and/or compositions comprising modified CHPs may be performed by an injection, a micro-dermal injection or topical application. In some embodiments, the administration of the modified CHPs and/or compositions comprising modified CHPs may be performed by application as a putty, a mesh, a patch, an adhesive, a cream, sutures or eyedrops.

Because the modified CHPs can include a variety of binding partners including various crosslinking motifs, the modified CHPs can be used for modulating cellular expression of a variety of cells through modification of their microenvironment and inducing specific cellular binding, or cytokine and enzymatic responses within the extracellular matrix. The term “cellular expression” refers to cellular expression of one or more proteins such as, for example, collagen, MMP and one or more extracellular matrix proteins. Accordingly, in an aspect of the present disclosure, a method for modulating cellular expression of certain types of cells may include contacting the modified CHPs to the corresponding types of cells. Examples of the cells in which cellular expression can be modulated using the modified CHPs described herein include, but are not limited to, osteocytes, tenocytes, chondrocytes, fibroblasts, osteoblasts, or mesenchymal stem cells (MSCs).

In some embodiments, modulating cellular expression may include decreasing a non-triple helical collagen concentration in microenvironment surrounding cells such as, for example, osteocytes, tenocytes, chondrocytes, fibroblasts, osteoblasts, or mesenchymal stem cells (MSCs). In some embodiments, modulating cellular expression may include increasing collagen expression in cells such as, for example, osteocytes, tenocytes, chondrocytes, fibroblasts, osteoblasts, or mesenchymal stem cells (MSCs).

In some embodiments, the modified CHPs can be contacted with the corresponding types of cells by administering the modified CHPs by local injection, intravenous injection, topical application, or physical application. In some embodiments, the administration of the modified CHPs may be performed by application as a putty, a mesh, a patch, an adhesive, a cream, sutures or eyedrops. In some embodiments, the modified CHPs are administered with a carrier such as, for example, collagen, extracellular matrix, artificial extracellular matrix, polymeric carries, protein carriers, mineral, glycosaminoglycans (GAGS), bioactive glass, liposome, and a mixture thereof.

### EXAMPLES

### Example 1

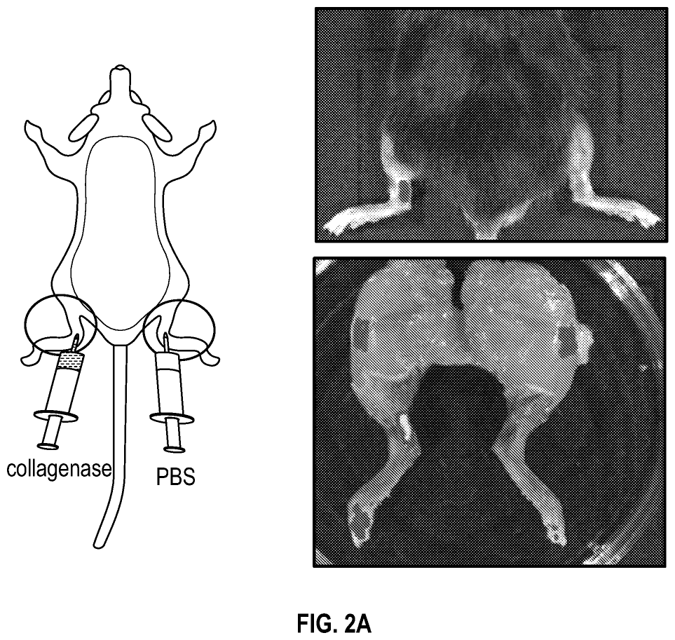

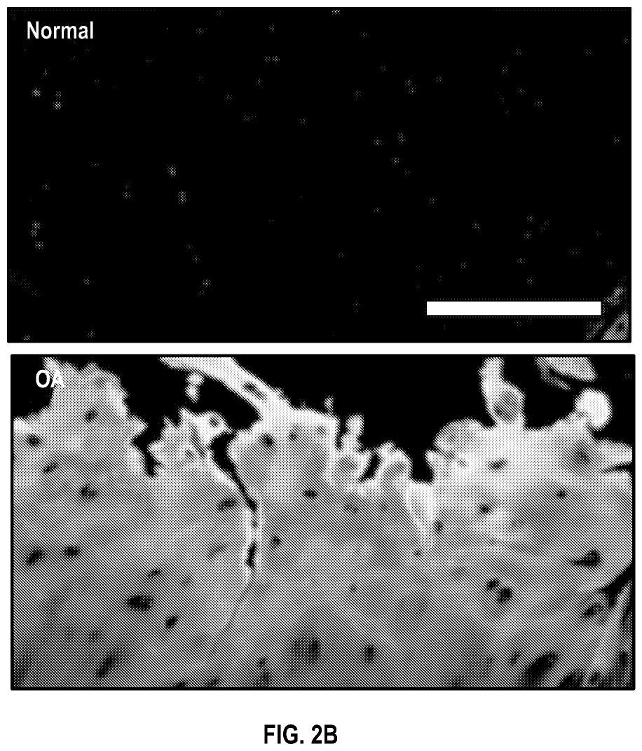

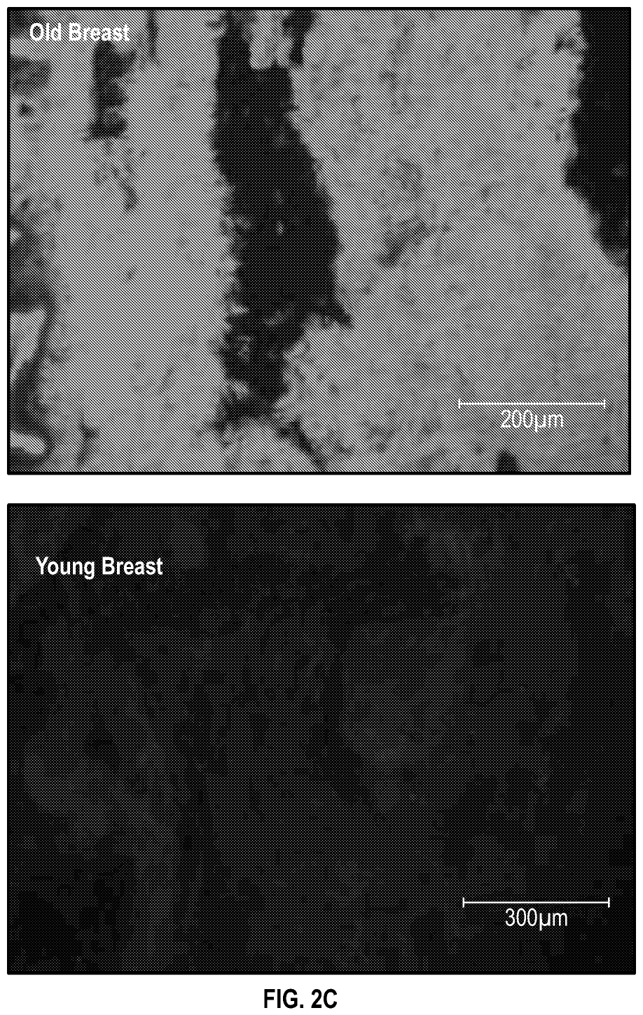

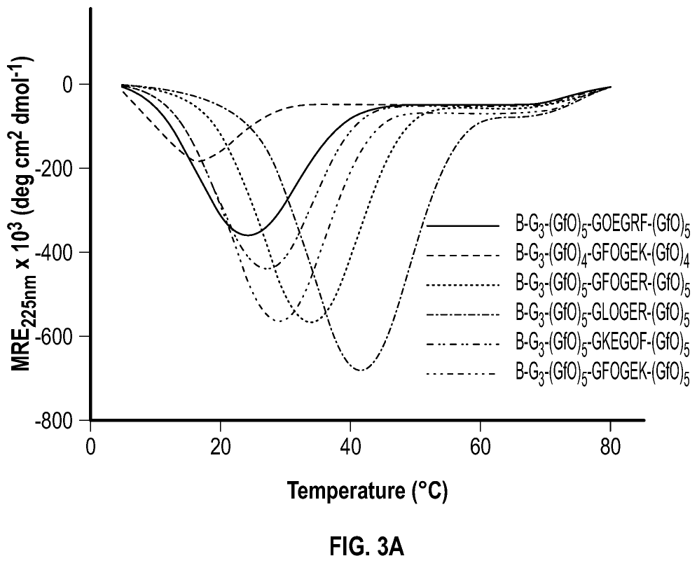

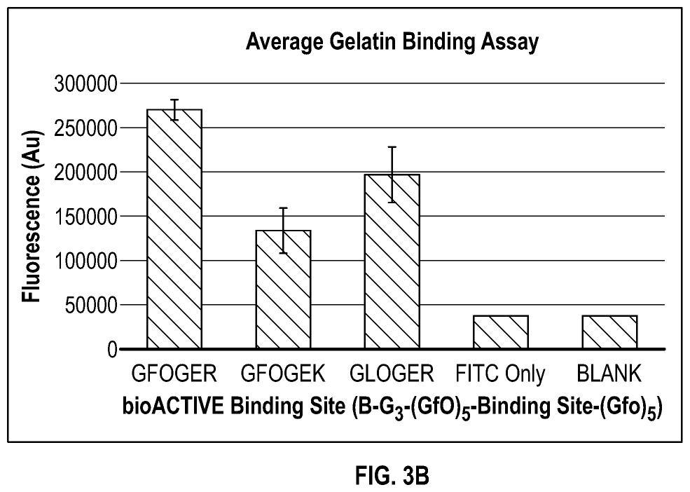

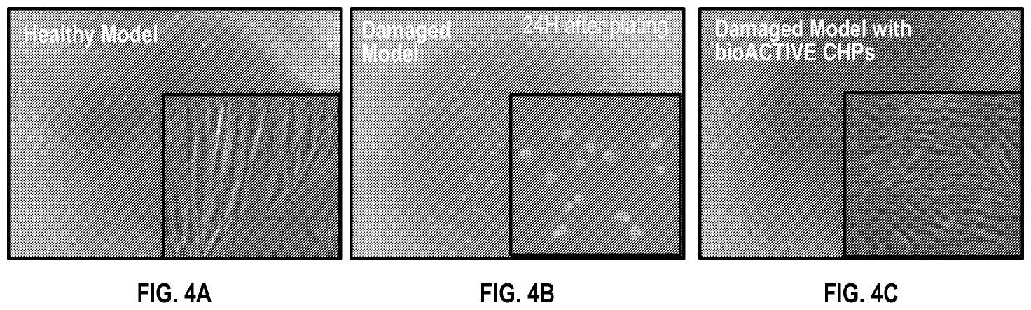

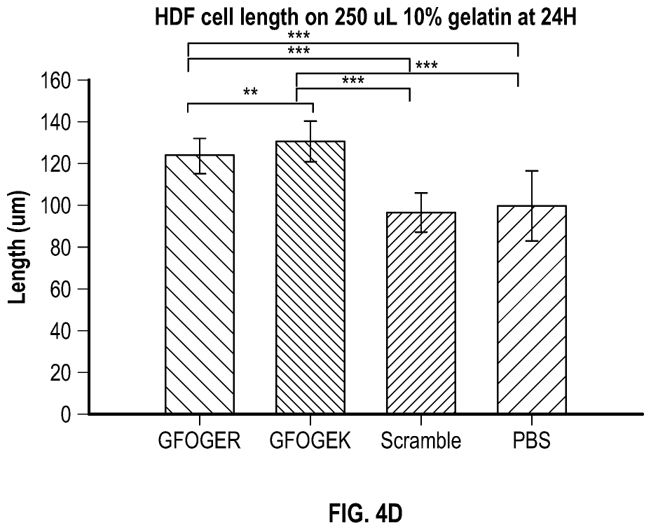

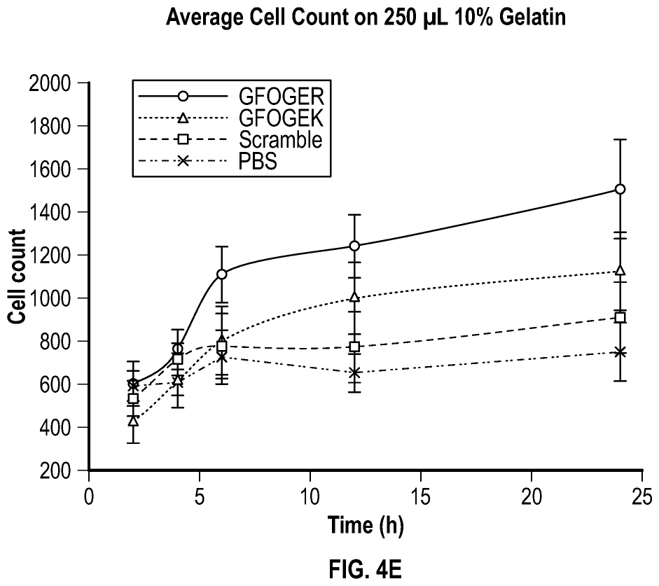

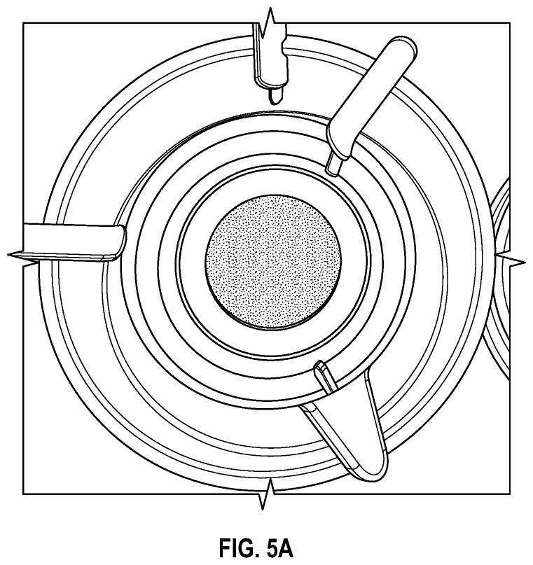

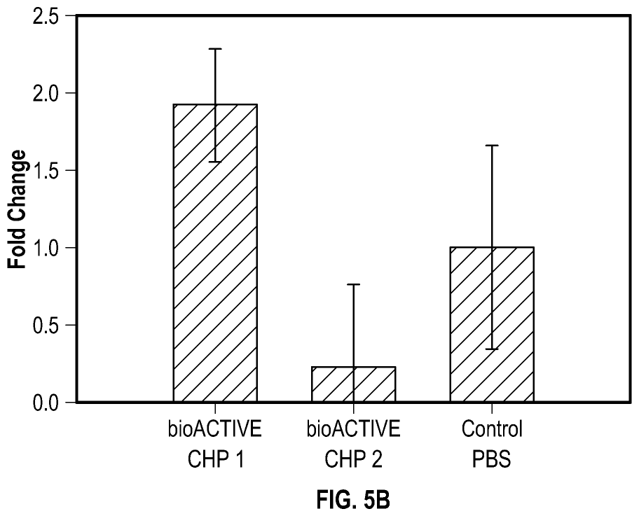

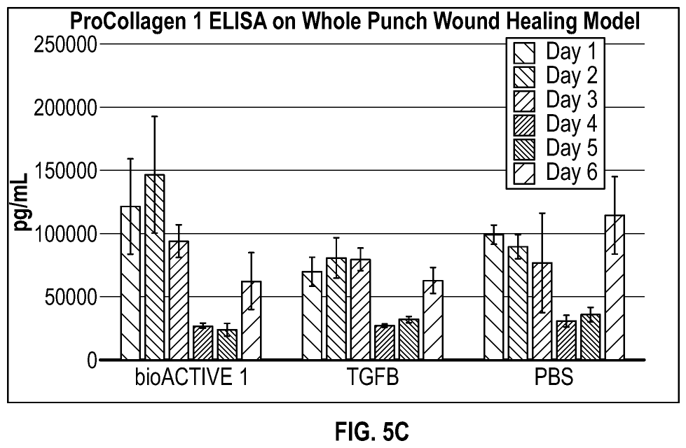

FIG. 2A shows CHPs targeting and binding to damaged collagen induced by collagenase activity in an in vivo mouse model of tendonitis.

C57BL/6J mouse model was used for testing CHPs on mouse tendons. Bacterial collagenase was used to create tendonitis model.

10 μl volume of 1 nmol CHP solution was injected in the mice via subcutaneous injections. The joints were imaged at specified time points (2 hr, 5 hr, 24 hr and 72 hr) and compared the Sham (PBS injected knee) vs the collagenase injected knee.

### Example 2

FIG. 2B shows CHPs targeting and binding to damaged collagen in human samples obtained from an osteoarthritic knee joint as compared to normal knee joint.

For formalin-fixed paraffin embedded (FFPE) sections from a knee joint (osteoarthritic knee joint as well as normal knee joint), a standard deparaffinization protocol was followed before applying CHP stock solution. CHP Stock solution was made to 20 μM and heated to 80° C. for 5 min. and quenched on ice for ˜60 sec prior to application to the tissue section. Volume was enough to cover the entire tissue (100-200 μl). Let CHPs stain overnight at 4° C. Then wash off 3×5 min with 1×PBS before counterstaining with DAPI.

### Example 3

FIG. 2C shows CHPs targeting and binding to damaged collagen in an aged human skin tissue as compared to young human skin tissue.

For Frozen sections OCT was washed out with 3×5 min washes with 1×PBS. CHP Stock solution was made to 20 μM and heated to 80° C. for 5 min then quickly quenched in ice water for ˜60 seconds prior to application to the tissue section. Volume was enough to cover the entire tissue (100-200 μl). Let CHPs stain overnight at 4° C. Then wash off 3×5 min with 1×PBS before counterstaining with DAPI.

### Example 4

FIG. 6 shows changes in gene expression caused by bioactive CHP disclosed herein.

IMR-90 human lung fibroblasts were purchased from ITCC. Cells were thawed and cultured in EMEM supplemented with 10% FBS and 1% Pen-Strep. Cells were cultured to 80-95% confluency then sub-cultured for three passages prior to utilization in experiments.

Experimental wells were prepared by adding 100 μL cell media to wells of a 96-well gelatin coated plate (corning) or 300 μL media to the wells of a 48-well uncoated cell culture plate. Peptides, as listed below, were added to each well (5 μL to 96 well plates, 10 μL to 48 well plates).

CF-Ahx-(GPO) 3-GFOGER-(GPO) 3-Heated, 2.5 μM

CF-Ahx-(GPO) 3-GFOGER-(GPO) 3-Unheated (triple helical), 2.5 μM

CF-Ahx-(GPO) 3-EGORFG-(GPO) 3-2.5 μM

Gelatin from porcine skin (30 μg/mL)

Collagen from bovine skin (30 μg/mL)

During passaging, cells were quantified, and 2100 IMR-90 cells were added to each well of the 96 well gelatin-coated plate. For the uncoated plate IMR-90 cells were added to each well. Additional media was added to bring the volume to 200 μL (96 well) or 500 μL (48 well). Cells were cultured at 37° C., 5% CO₂ for three days then imaged. IMR-90 cells were mostly confluent with wells that had gelatin or collagen added to media appeared to have higher confluence and more directionality in cell growth. Media was added one day later (day 4) then allowed to culture an additional 2 days before imaging and lysing cells. qPCR was performed on cell lysate following cDNA synthesis (cell-to-CT kit, Thermo Fisher scientific).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A modified collagen hybridizing peptide (CHP) comprising one or more binding partners crosslinked to a CHP, wherein the CHP has a sequence represented by Formula I:
(Gly-X-Y)_{a-b} (Formula I)
in which Gly is glycine; at least one of X and Y is proline, modified proline, and/or hydroxyproline; and a is 3 and b is 20,
wherein the one or more binding partner is selected from the group consisting of integrin sites, integrin binding sites, crosslinking sites, von Willebrand Factor (VWF) discoidin domain receptor (DDR) 1, DDR 2, SPARC binding peptides, fibronectin binding peptides, engineered integrin binding peptides, matrix metalloproteinase (MMP) cleavage sites, Cathepsin K (CATK) sites, and osteoclast-associated receptors (OSCAR).

2. The modified CHP according to claim 1, wherein the one or more binding partner comprises an integrin site.

3. The modified CHP according to claim 1, wherein the one or more binding partner comprises an integrin binding site.

4. The modified CHP according to claim 1, wherein the one or more binding partner comprises an integrin binding site comprising a sequence having at least 85% sequence identity to any one of SEQ ID NO: 1-16.

5. The modified CHP according to claim 1, wherein the integrin binding site comprises an integrin α1β1.

6. The modified CHP according to claim 1, wherein the integrin binding site comprises an integrin α2β1.

7. The modified CHP according to claim 1, wherein the integrin binding site comprises an integrin α10β1.

8. The modified CHP according to claim 1, wherein the integrin binding site comprises an integrin α11β1.

9. The modified CHP according to claim 1, the one or more binding partner comprises a crosslinking site.

10. The modified CHP according to claim 1, the one or more binding partner comprises a sugar crosslinking site.

11. The modified CHP according to claim 1, the one or more binding partner comprises a crosslinking site comprising a sequence having at least 85% sequence identity to SEQ ID NO: 17.

12. The modified CHP according to claim 1, wherein the one or more binding partner comprises a VWF.

13. The modified CHP according to claim 1, wherein the one or more binding partner comprises a DDR 1, DDR 2, or SPARC binding peptide.

14. The modified CHP according to claim 1, wherein the one or more binding partner comprises a VWF binding peptide comprising a sequence having at least 85% sequence identity to any one of SEQ ID NO: 18-21.

15. The modified CHP according to claim 1, wherein the one or more binding partner comprises a DDR1, DDR 2 or SPARC binding peptide comprising a sequence having at least 85% sequence identity to any one of SEQ ID NO: 18-21.

16. The modified CHP according to claim 1, wherein the one or more binding partner comprises a fibronectin binding motif or sequence.

17. The modified CHP according to claim 1, wherein the one or more binding partner comprises a fibronectin binding motif comprising a sequence having at least 85% sequence identity to SEQ ID NO: 22 or 23.

18. The modified CHP according to claim 1, wherein the one or more binding partner comprises an engineered integrin binding motif.

19. The modified CHP according to claim 1, wherein the one or more binding partner comprises an engineered integrin binding motif comprising a sequence having at least 85% sequence identity to SEQ ID NO: 24 or 25.

20. The modified CHP according to claim 1, wherein the one or more binding partner comprises an MMP cleavage site.

21. The modified CHP according to claim 1, wherein the one or more binding partner comprises an MMP cleavage site comprising a sequence having at least 85% sequence identity to SEQ ID NO: 26 or 27.

22. The modified CHP according to claim 1, wherein the CHP has a sequence selected from the group consisting of SEQ ID NO: 1-169.

23. The modified CHP according to claim 1, wherein the modified CHP has one binding partner, and said one binding partner is crosslinked to C-terminus of the CHP.

24. The modified CHP according to claim 1, wherein the modified CHP has one binding partner, and said one binding partner is crosslinked to N-terminus of the CHP.

25. The modified CHP according to claim 1, wherein the modified CHP has two binding partners, one of said two binding partners is crosslinked to C-terminus of the CHP, and the other one of said two binding partners is crosslinked to N-terminus of the CHP.

26. The modified CHP according to claim 1, wherein the one or more binding partners is attached to the CHP at N-terminus and is attached to another CHP at C-terminus.

27. The modified CHP according to claim 1, wherein said one or more binding partners are crosslinked to a CHP directly.

28. The modified CHP according to claim 1, wherein said one or more binding partners are crosslinked to a CHP via one or more spacer.

29. The modified CHP according to claim 1, wherein the modified CHP comprises a cap at the N-terminus of the modified CHP.

30. The modified CHP according to claim 1, wherein the cap comprises an acetyl group (Ac).

31. The modified CHP according to claim 29, wherein the CHP is crosslinked to the cap directly.

32. The modified CHP according to claim 29, wherein the modified CHP comprises one or more spacer between the cap and the CHP.

33. The modified CHP according to claim 29, wherein the modified CHP comprises one or more spacer between the cap and the one or more binding partners.

34. The modified CHP according to claim 29, wherein the one or more binding partners are crosslinked to the cap directly.

35. The modified CHP according to claim 29, wherein the CHP comprises the cap- the spacer-CHP-binding partner-CHP.

36. A composition comprising the modified CHP of claim 1.

37. The composition according to claim 36, wherein the modified CHP does not form a triple helix with other modified CHPs.

38. The composition according to claim 36, further comprising a carrier.

39. The composition according to claim 36, wherein the composition is a cosmetic composition, and the carrier comprises at least one selected from the group consisting of micelles, dendrites, lipids, microemulsions, nanoemulsions, solid lipid nanoparticles, nanostructured lipid carriers, liposomes, transfersomes, ethosomes, niosomes, collagen matrix, extracellular matrix, and artificial extracellular matrix.

40. A method of increasing collagen production in a subject, comprising administering the modified CHP of the cosmetic composition of claim 36 to the subject.

41. The method according to claim 40, wherein the administering is performed by injection, micro-dermal injection, or topical application.

42. A method of decreasing MMP production in a subject, comprising administering the modified CHP of the cosmetic composition of claim 36 to the subject.

43. The method according to claim 42, wherein the administering is performed by injection, micro-dermal injection, or topical application.

44. A method of inducing dermal repair in a subject, comprising administering the cosmetic composition of claim 36 to the subject.

45. The method according to claim 44, wherein the administering is performed by injection, micro-dermal injection, or topical application.

46. A method of modulating cellular expression of osteocytes, tenocytes, chondrocytes, fibroblasts, osteoblasts, or mesenchymal stem cells (MSCs), the method comprising contacting the modified CHP of claim 35 to any one of osteocytes, tenocytes, chondrocytes, fibroblasts, osteoblasts and MSCs.

47. The method of according to claim 46, wherein the cellular expression comprises one or more expressions of collagen, MMP, and extracellular matrix protein.

48. The method according to claim 46, wherein the method comprises decreasing a non-triple helical collagen concentration in microenvironment surrounding said osteocytes, tenocytes, chondrocytes, fibroblasts, osteoblasts, or MSCs.

49. The method according to claim 46, wherein the method comprises increasing collagen expression in said osteocytes, tenocytes, chondrocytes, fibroblasts, osteoblasts, or MSCs.

50. The method according to claim 46, wherein the administering is performed by local injection, intravenous injection, topical application, or physical application.

51. The method according to claim 46, wherein the modified CHP is administered as a putty, mesh, patch, adhesive, cream, sutures, or eyedrops.

52. The method according to claim 46, wherein the modified CHP is administered with a carrier.

53. The method according to claim 52, wherein the carrier comprises at least one selected from the group consisting of collagen, extracellular matrix, artificial extracellular matrix, polymeric carries, protein carriers, mineral, glycosaminoglycans (GAGS), bioactive glass, liposome, and a mixture thereof.
